(19) United States
(12) Patent Application Publication
(10) Pub. No.: US 2012/0217485 A1
(43) Pub. Date: Aug. 30, 2012

(54) NOVEL ORGANIC ELECTROLUMINESCENT COMPOUNDS AND ORGANIC ELECTROLUMINESCENT DEVICE USING THE SAME

(75) Inventors: Hyo Jung Lee, Geumcheon-gu (KR); Young Jun Cho, Seongbuk-gu (KR); Sung Jin Eum, Guro-gu (KR); Hyuck Joo Kwon, Dongdaemun-gu (KR); Bong Ok Kim, Gangnam-gu (KR); Sung Min Kim, Yangcheon-gu (KR); Seung Soo Yoon, Suwon-si (KR)

(73) Assignee: ROHM AND HAAS ELECTRONIC MATERIALS KOREA LTD., CHUNGCHEONGNAM-DO (KR)

(21) Appl. No.: 13/388,244

(22) PCT Filed: Jul. 30, 2010
(86) PCT No.: PCT/KR10/05039

§ 371 Date: May 2, 2012

(30) Foreign Application Priority Data
Jul. 31, 2009 (KR) 10-2009-007061
Jul. 6, 2010 (KR) 10-2010-0064849

(51) Int. Cl. H01L 51/50; C07D 413/04; C07D 417/04; C07D 403/14

(52) U.S. Cl. 257/40; 544/284; 544/102; 544/42; 540/587; 257/E51.019

(57) ABSTRACT

Provided are novel organic electroluminescent compounds and organic electroluminescent devices using the same. Since the organic electroluminescent compound exhibits good luminous efficiency and excellent life property compared to the existing material, it may be used to manufacture OLED devices having superior operation life and consuming less power due to improved power efficiency.

### FIELD OF THE INVENTION

The present invention relates to novel organic electroluminescent compounds and an organic electroluminescent device using the same, more particularly, to novel organic electroluminescent compounds used as an electroluminescent material and an organic electroluminescent device using the same as a host.

### BACKGROUND OF THE INVENTION

At present, CBP is the most widely known as a host material for a phosphorescent material. High-efficiency OLEDs using a hole blocking layer including BCP and BAlq and high-performance OLEDs using BAlq derivatives as a host were reported.

Although these materials provide good electroluminescence characteristics, they are disadvantageous in that degradation may occur during the high-temperature deposition process in vacuum because of low glass transition temperature and poor thermal stability. Since the power efficiency of an OLED is given by (π/voltage)×current efficiency, the power efficiency is inversely proportional to the voltage. High power efficiency is required to reduce the power consumption of an OLED. Actually, OLEDs using phosphorescent materials provide much better current efficiency (cd/A) than those using fluorescent materials. However, when the existing materials such as BAlq, CBP, etc. are used as a host of the phosphorescent material, there is no significant advantage in power efficiency (1 m/W) over the OLEDs using fluorescent materials because of high driving voltage.

In addition, the OLED devices do not have satisfactory operation life. Therefore, development of more stable and higher-performance host materials is required.

### DISCLOSURE

### Technical Problem

With intensive efforts to overcome the problems of conventional techniques as described above, the present inventors have invented novel organic electroluminescent compounds which realize organic electroluminescent devices having excellent luminous efficiency and noticeably improved life property.

The object of the present invention is to provide organic electroluminescent compounds having the backbone to provide better luminous efficiency and device life with appropriate color coordinate as compared to conventional host or dopant material, while overcoming the problems described above. Another object is to provide a highly efficient and long life organic electroluminescent device using the organic electroluminescent compounds.

### Technical Solution

Provided are novel organic electroluminescent compounds represented by Chemical Formula 1 and an organic electroluminescent device using the same. Since the organic electroluminescent compound according to the present invention exhibits good luminous efficiency and excellent life property compared to the existing host material, it may be used to manufacture OLED devices having very superior operation life.

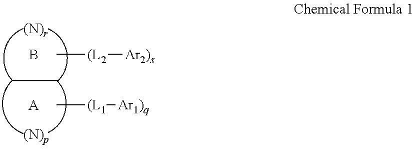

wherein
a ring A and a ring B represent a 6-membered aromatic ring or a 6-membered hetero aromatic ring;
p and r represent an integer from 0 to 2, and q and s represent an integer from 2 to 4, with the proviso that p+r≧1, p+q=4 and r+s=4;
L₁ and L₂ independently represent a chemical bond, (C6-C30)arylene with or without substituent(s), (C3-C30)heteroarylene with or without substituent(s), 5- to 7-membered heterocycloalkylene with or without substituent(s), 5- to 7-membered heterocycloalkylene fused with one or more aromatic ring(s) with or without substituent(s), (C3-C30)cycloalkylene with or without substituent(s), (C3-C30)cycloalkylene fused with one or more aromatic ring(s) with or without substituent(s), (C2-C30)alkenylene with or without substituent(s), (C2-C30)alkynylene with or without substituent(s), (C6-C30)ar(C1-C30)alkylene with or without substituent(s), —O— or —S—;
Ar₁ and Ar₂ independently represent hydrogen, halogen, (C1-C30)alkyl with or without substituent(s), (C6-C30)aryl with or without substituent(s), substituted or unsubstituted (C6-C30)aryl fused with one or more (C3-C30)cycloalkyl with or without substituent(s), (C3-C30)heteroaryl with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), cyano, —NR₁₁R₁₂, —SiR₁₃R₁₄R₁₅, —OR₁₆, —SR₁₇, (C6-C30) ar (C1-C30) alkyl with or without substituent(s), (C1-C30)alkylamino with or without substituent(s), (C3-C30)heteroaryl with or without —SiR₁₈R₁₉R₂₀ substituent(s), (C6-C30)arylamino with or without substituent(s), (C2-C30)alkenyl with or without substituent(s), (C2-C30)alkynyl with or without substituent(s), carboxyl, nitro,

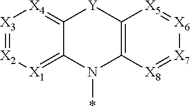

or hydroxyl, or each of them may be linked to an adjacent substituent via (C3-C30)alkylene or (C3-C30)alkenylene with or without a fused ring to form an alicylic ring or a mono- or polycyclic aromatic ring;
the R₁₁ through R₂₀ independently represent (C1-C30)alkyl with or without substituent(s), (C6-C30)aryl with or without substituent(s) or (C3-C30)heteroaryl with or without substituent(s) or each of them may be linked to an adjacent substituent via (C3-C30)alkylene or (C3-C30)alkenylene with or without a fused ring to form an alicylic ring or a mono- or polycyclic aromatic ring;
the X₁ through X₈ independently represent CR₂₁ or N; except for the case where all of X₁ through X₈ are CR₂₁ or N;
Y represents a chemical bond, —(CR₂₂R₂₃)ₘ—, —(R₂₄)C═C(R₂₅)—, —N(R₂₆)—, —S—, —O— or —Si(R₂₇)(R₂₈)—, R₂₁ through R₂₈ are the same as defined in the R₁₁ through R₂₀, and m represents an integer of 1 or 2;
the heterocycloalkyl or heteroaryl may include one or more heteroatom(s) selected from N, O, S and Si.

In the present invention, “alkyl”, “alkoxy” and other substituents including “alkyl” moiety include both linear and branched species and “cycloalkyl” includes monocyclic hydrocarbons as well as polycyclic hydrocarbons such as adamantyl or bicycloalkyl. The term “aryl” described herein means an organic radical derived from aromatic hydrocarbon via elimination of one hydrogen atom. Each ring includes a monocyclic or fused ring system including from 4 to 7, preferably from 5 to 6 cyclic atoms. A structure that one or more aryls are linked by a chemical bond is also included. Specific examples of the aryl include phenyl, naphthyl, biphenyl, anthryl, indenyl, fluorenyl, phenanthryl, triphenylenyl, pyrenyl, perylenyl, chrysenyl, naphthacenyl and fluoranthenyl, but they are not restricted thereto.

The term “heteroaryl” described herein means an aryl group including from 1 to 4 heteroatom(s) selected from N, O and S for the aromatic cyclic backbone atoms, and carbon atom(s) for remaining aromatic cyclic backbone atoms. The heteroaryl may be a 5- or 6-membered monocyclic heteroaryl or a polycyclic heteroaryl which is fused with one or more benzene ring(s), and may be partially saturated. In addition, the term “heteroaryl” described herein includes a structure that one or more heteroaryls are linked by a chemical bond.

The heteroaryl groups may include divalent aryl groups of which the heteroatoms are oxidized or quarternized to form N-oxides, quaternary salts, or the like. Specific examples of the heteroaryl include monocyclic heteroaryl groups such as furyl, thienyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups such as benzofuryl, benzothienyl, isobenzofuryl, benzimidazolyl, benzothiazolyl, benzisothiazolyl, benzisoxazolyl, benzoxazolyl, isoindolyl, indolyl, indazolyl, benzothiadiazolyl, quinolyl, isoquinolyl, cinnolinyl, quinazolinyl, quinolizinyl, quinoxalinyl, carbazolyl, phenanthridinyl and benzodioxolyl; and corresponding N-oxides (for example, pyridyl N-oxide, quinolyl N-oxide) and quaternary salts thereof; but they are not restricted thereto.

The “(C1-C30)alkyl” groups described herein may include (C1-C20)alkyl or (C1-C10)alkyl and the “(C6-C30)aryl” groups include (C6-C20)aryl or (C6-C12)aryl. The “(C3-C30)heteroaryl” groups include (C3-C20)heteroaryl or (C3-C12)heteroaryl and the “(C3-C30)cycloalkyl” groups include (C3-C20)cycloalkyl or (C3-C7)cycloalkyl. The “(C2-C30)alkenyl or alkynyl” group include (C2-C20)alkenyl or alkynyl, (C2-C10)alkenyl or alkynyl.

In “with or without substituents” of the R₁₁ through R₂₀, L₁, L₂, Ar₁, Ar₂, and R₂₁ through R₂₈, the substituent is further substituted by one or more substituent(s) independently selected from deuterium, halogen, (C1-C30)alkyl with or without halogen substituent(s), (C6-C30)aryl, (C3-C30)heteroaryl with or without (C6-C30)aryl substituent(s), 5- to 7-membered heterocycloalkyl, 5- to 7-membered heterocycloalkyl fused with one or more aromatic rings, (C3-C30)cycloalkyl, (C3-C30)cycloalkyl fused with one or more aromatic rings, tri(C1-C30)alkylsilyl, di(C1-C30)alkyl(C6-C30)arylsilyl, tri(C6-C30)arylsilyl, (C2-C30)alkenyl, (C2-C30)alkynyl, cyano, carbazolyl, —NR₃₁R₃₂, —SiR₃₃R₃₄R₃₅, —OR₃₆, —SR₃₇, (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, carboxyl, nitro or hydroxyl, or is linked to an adjacent substituent to form a ring,
the R₃₁ through R₃₇ are independently linked to (C1-C30)alkyl, (C6-C30)aryl, (C3-C30)heteroaryl or an adjacent substituent via (C3-C30)alkylene or (C3-C30)alkenylene with or without a fused ring to form an alicylic ring or a mono- or polycyclic aromatic ring.

The R₁₁ through R₂₈ are selected from hydrogen, halogen, alkyl such as methyl, ethyl, propyl, butyl, pentyl, hexyl, ethylhexyl, heptyl, octyl, etc., aryl such as phenyl, naphthyl, fluorenyl, biphenyl, phenanthryl, terphenyl, pyrenyl, perylenyl, spirobifluorenyl, fluoranthenyl, chrysenyl, triphenylenyl, etc., aryl fused with one or more cycloalkyl such as 1,2-dihydroacenaphthyl, heteroaryl such as dibenzothiophenyl, dibenzofuryl, carbazolyl, pyridyl, furyl, thienyl, quinolyl, triazinyl, pyrimidinyl, pyridazinyl, quinoxalinyl, phenanthrolinyl, etc., heterocycloalkyl fused with one or more aromatic ring such as benzopyrrolidino, benzopiperidino, dibenzomorpholino, dibenzoazepino, etc., amino substituted by aryl such as phenyl, naphthyl, fluorenyl, biphenyl, phenanthryl, terphenyl, pyrenyl, perylenyl, spirobifluorenyl, fluoranthenyl, chrysenyl, triphenylenyl, etc. or heteroaryl such as dibenzothiophenyl, dibenzofuryl, carbazolyl, pyridyl, furyl, thienyl, quinolyl, triazinyl, pyrimidinyl, pyridazinyl, quinoxalinyl, phenanthrolinyl, etc., aryloxy such as biphenyloxy, etc., arylthio such as biphenylthio, etc., aralkyl such as biphenylmethyl, triphenylmethyl, etc., carboxyl, nitro, or hydroxyl, but are not limited thereto, and may be further substituted as shown in Chemical Formula 1.

In addition, the organic electroluminescent compound according to the present invention may be exemplified as compounds having following structures represented by Chemical Formulas 2 to 5 but is not limited thereto.

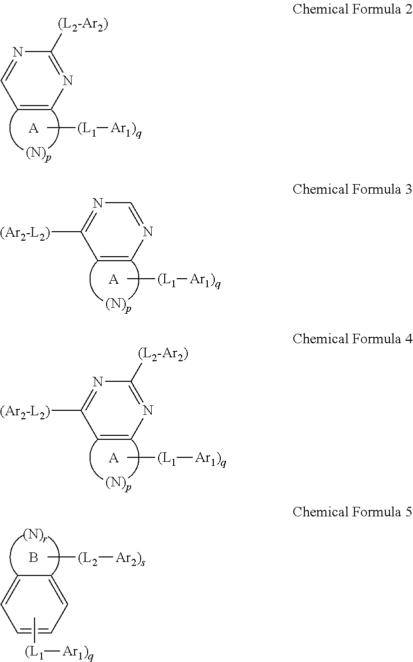

wherein
a ring A, a ring B, Ar₁, Ar₂, p, q, r and s are the same as defined in Chemical Formula 1, wherein each of the substituents may be different from each other.

The organic electroluminescent compound according to the present invention may be exemplified as compounds having following structures but is not limited thereto.

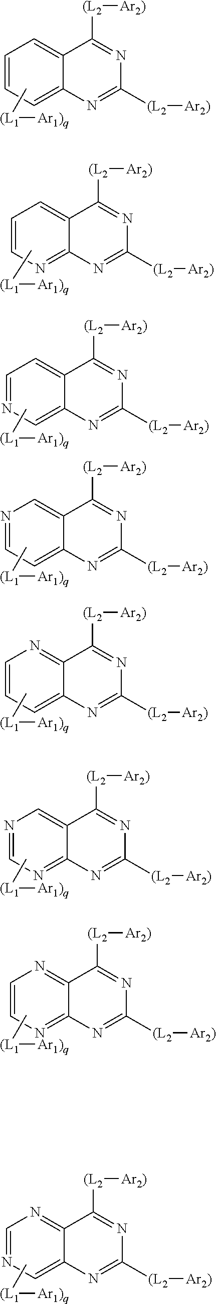

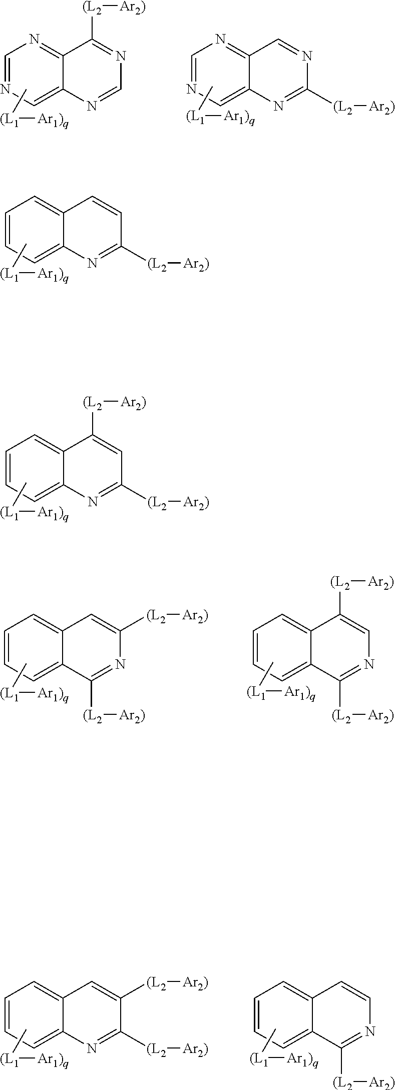

wherein

L₁, L₂, Ar₁, Ar₂ and q are the same as defined in Chemical Formula 1, wherein each of the substituents may be different from each other.

The Ar₁and Ar₂ according to the present invention may be exemplified as following structures but are not limited thereto.

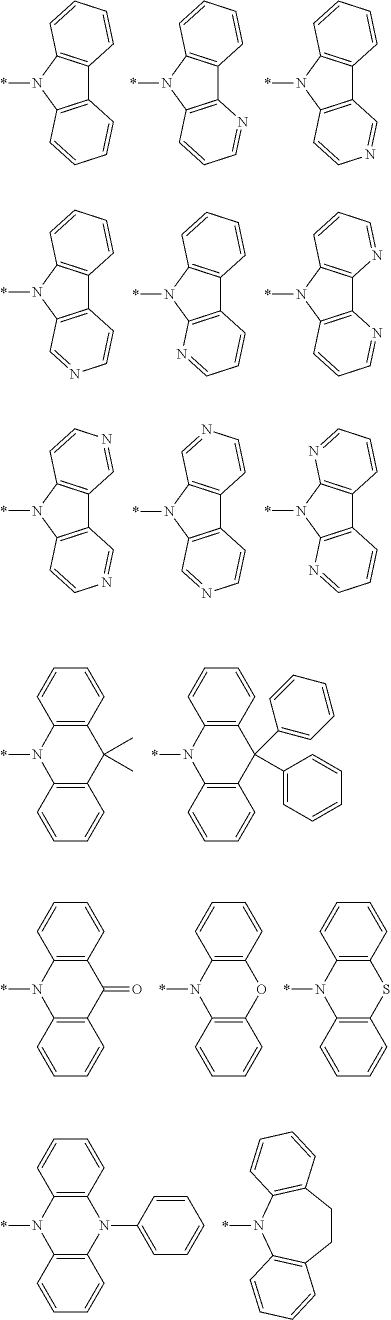

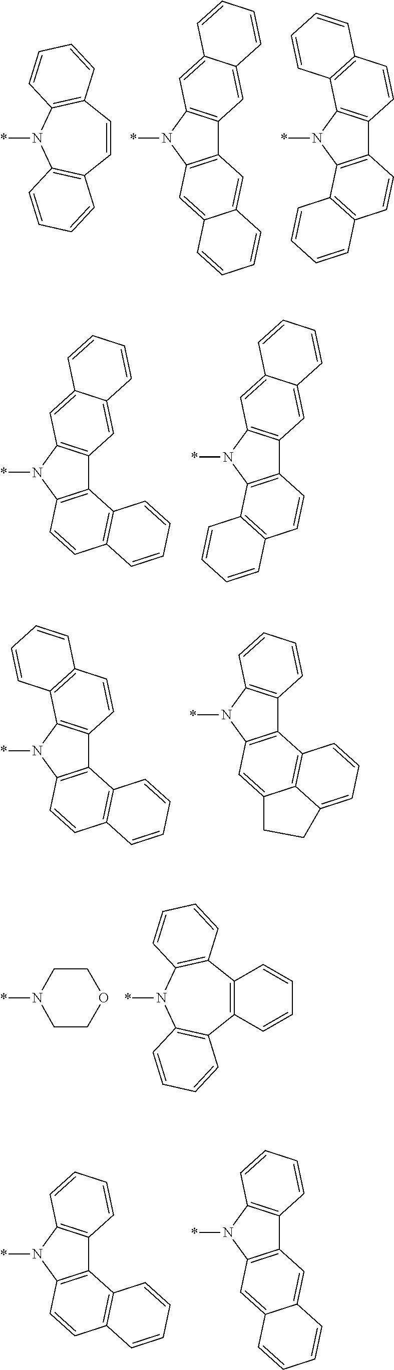

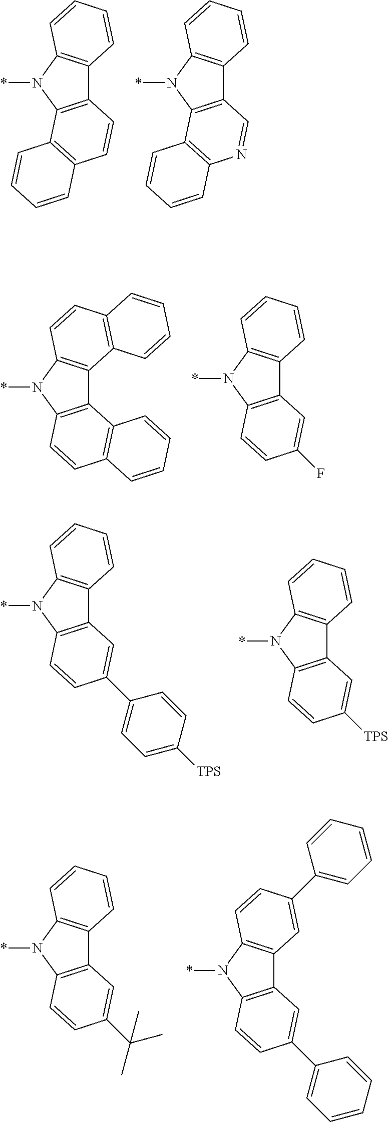

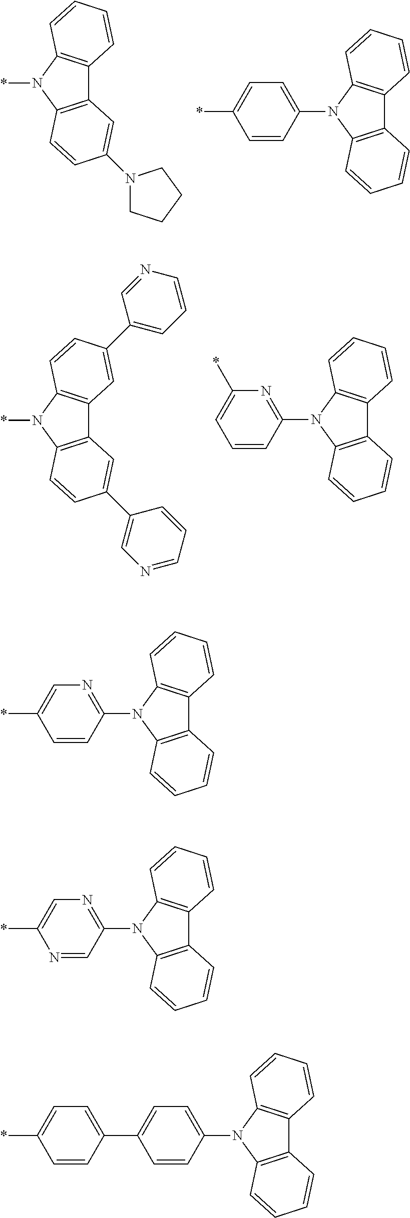

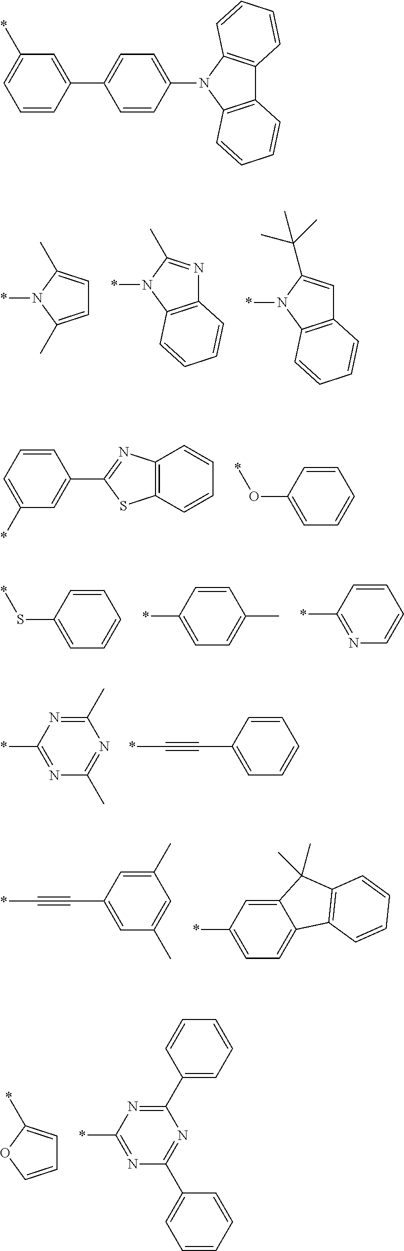

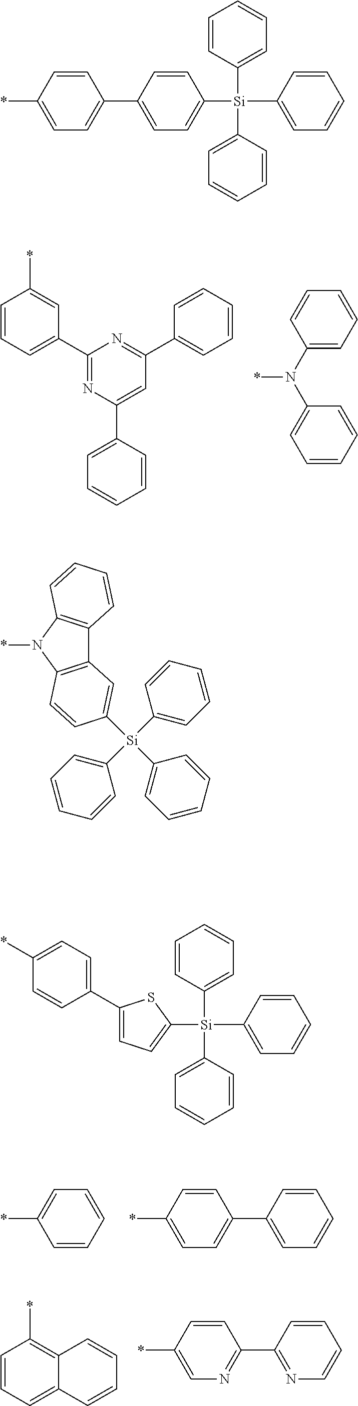

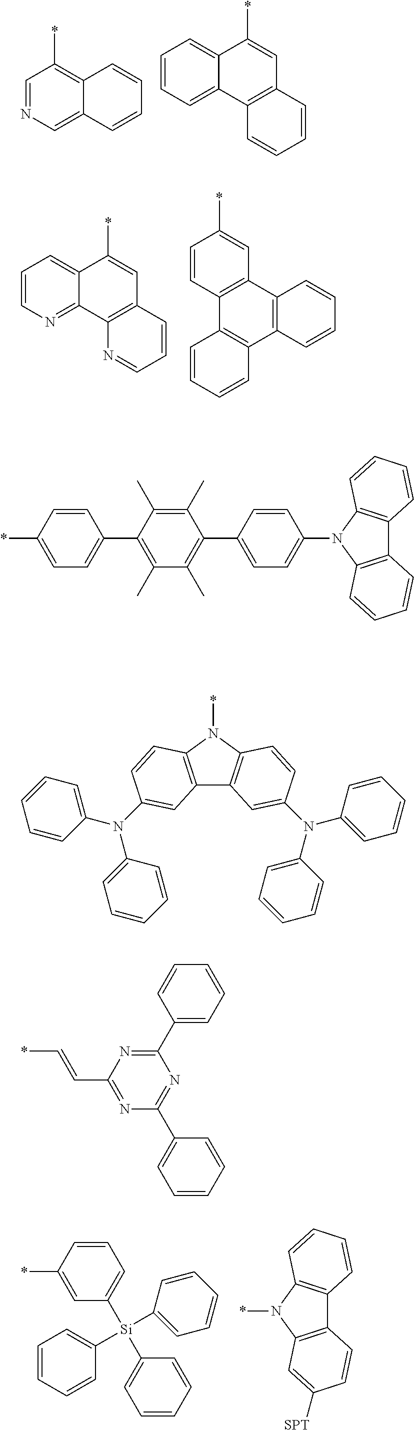

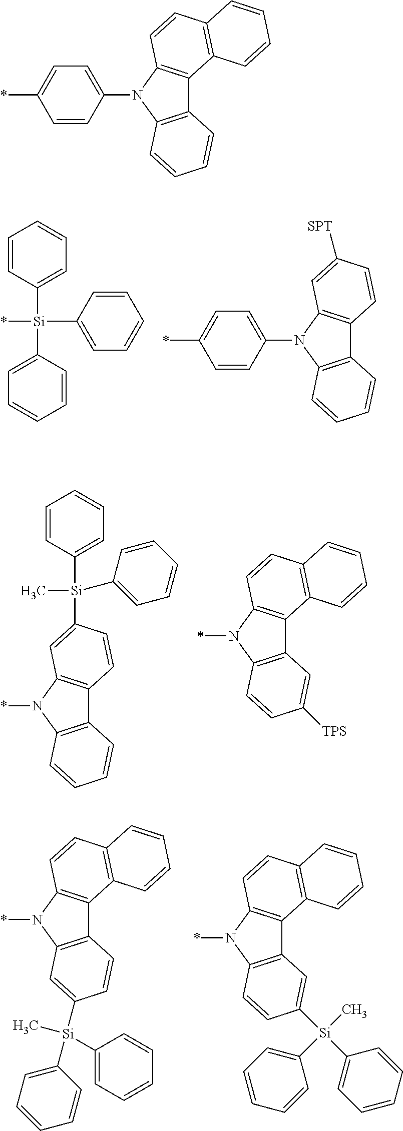

The organic electroluminescent compound according to the present invention may be more specifically exemplified as following compounds but is not limited thereto.

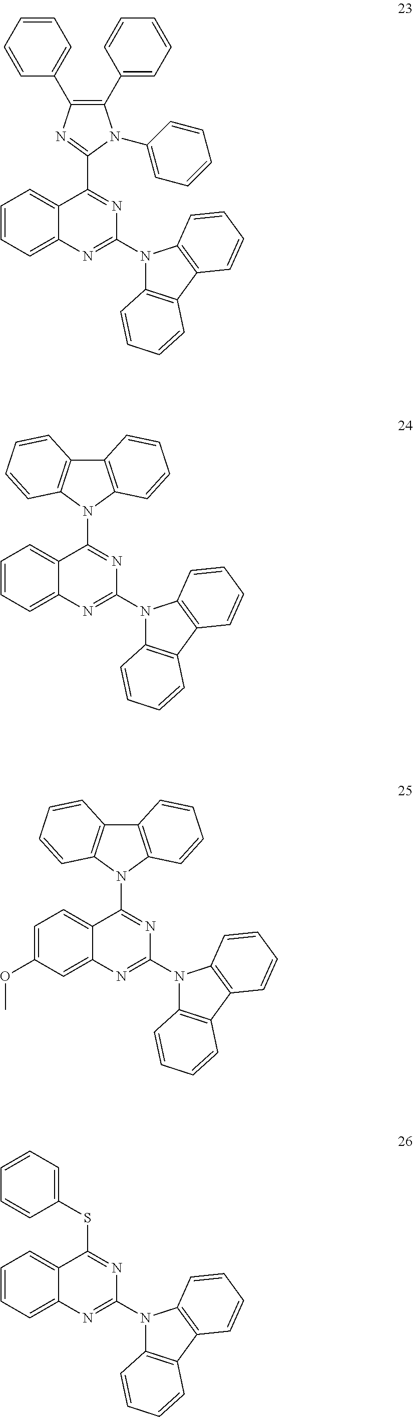

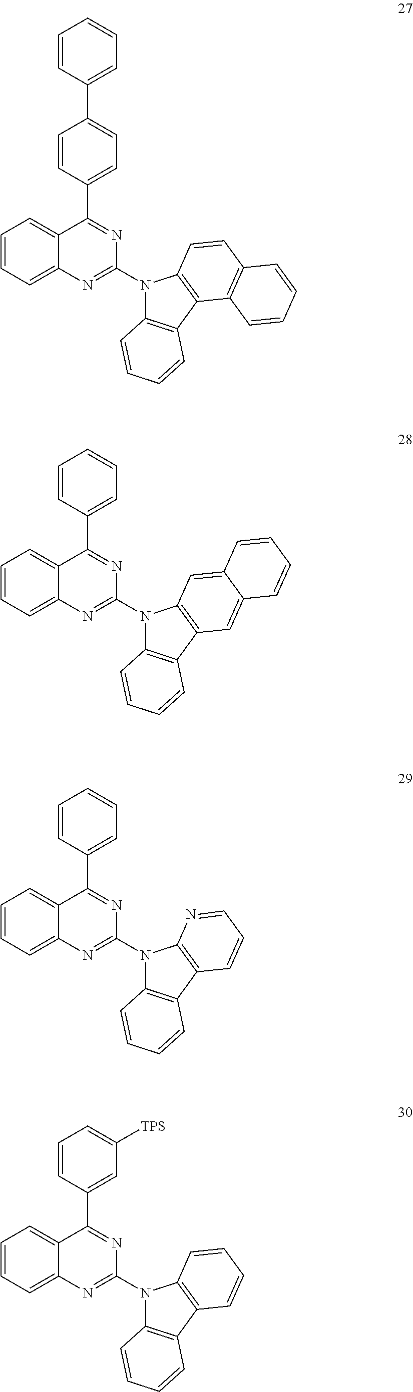

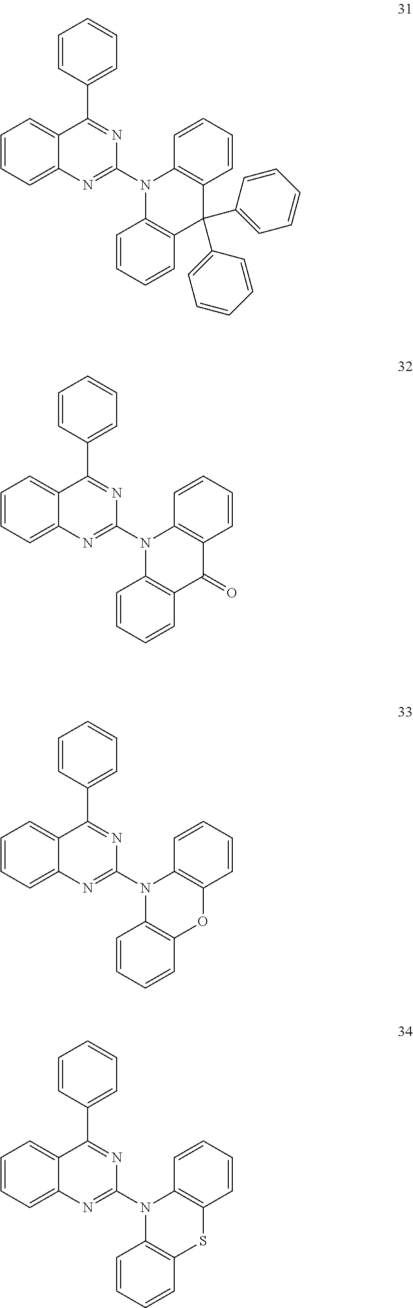

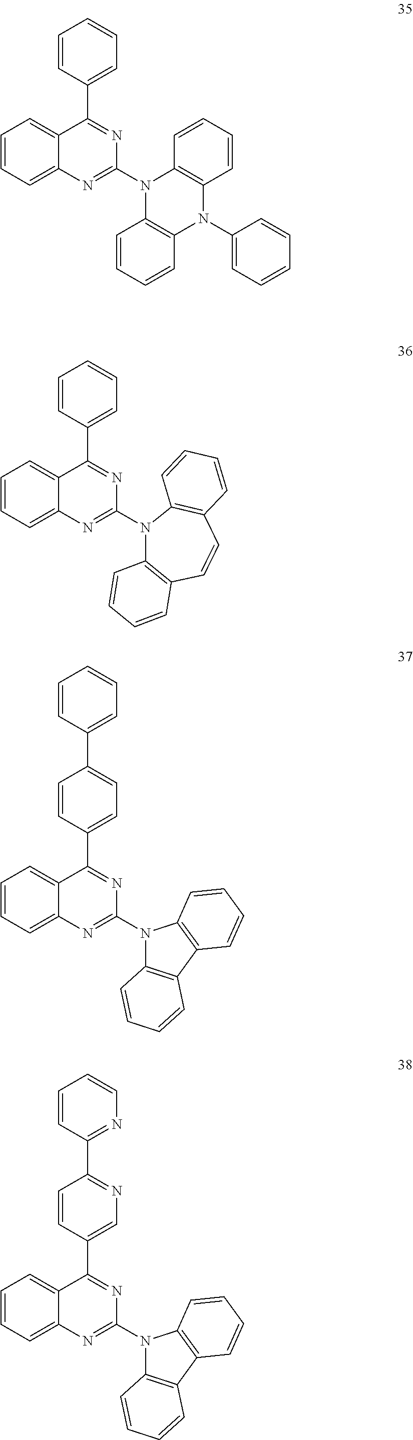

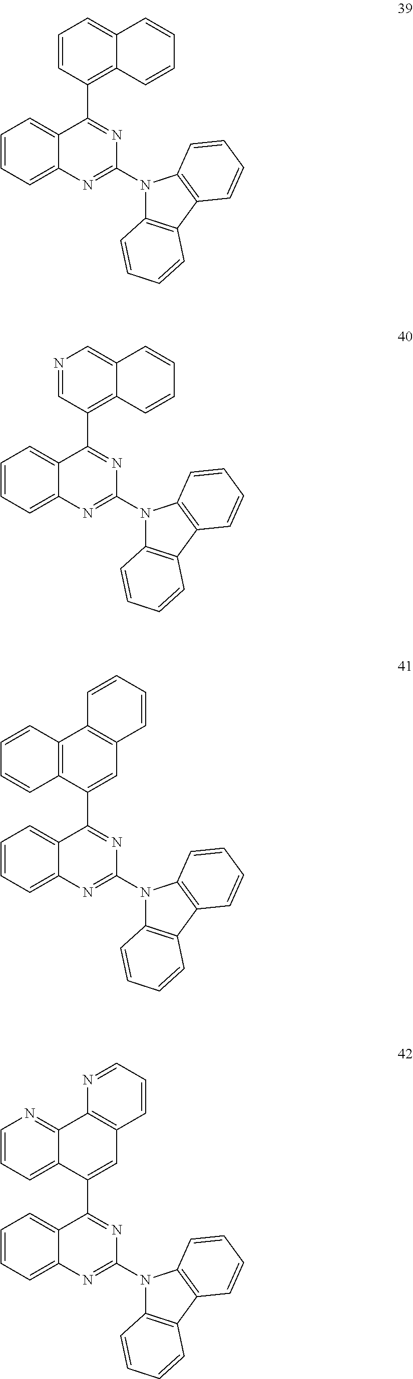

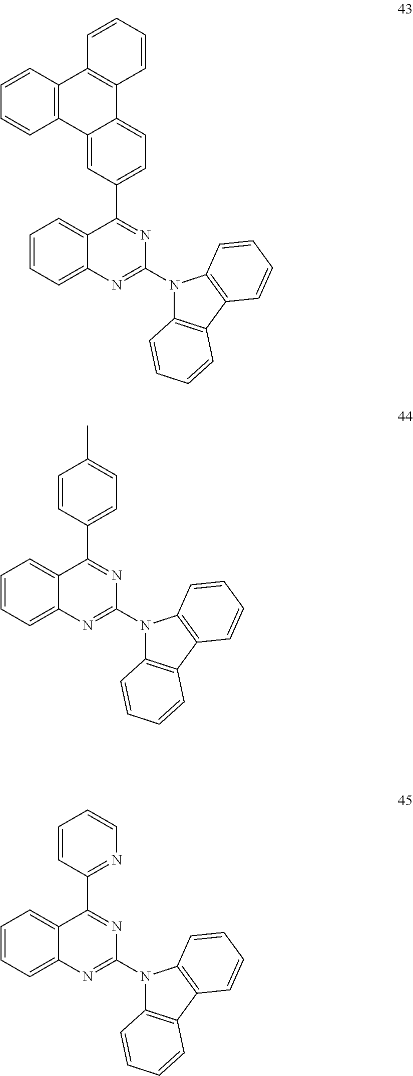

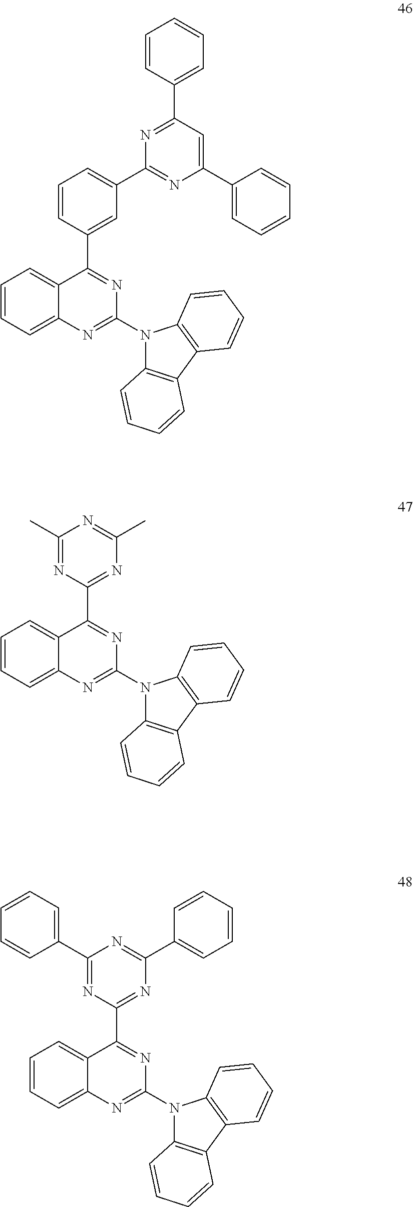

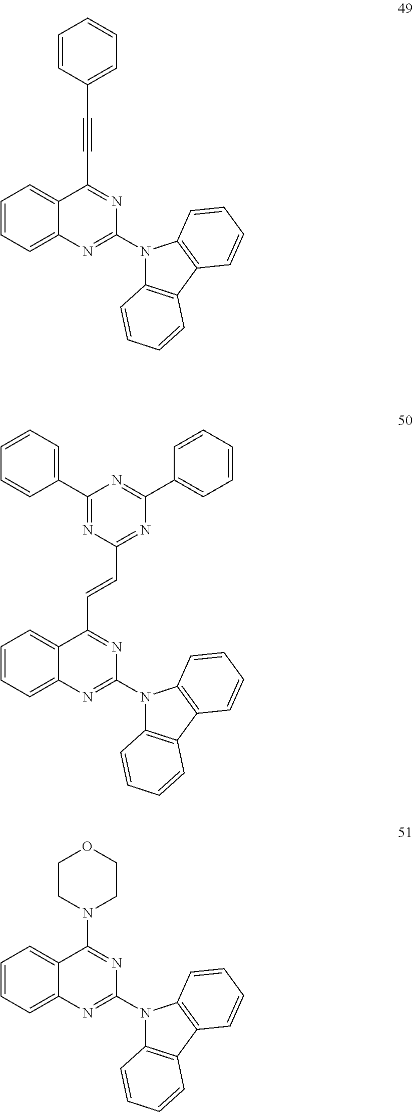

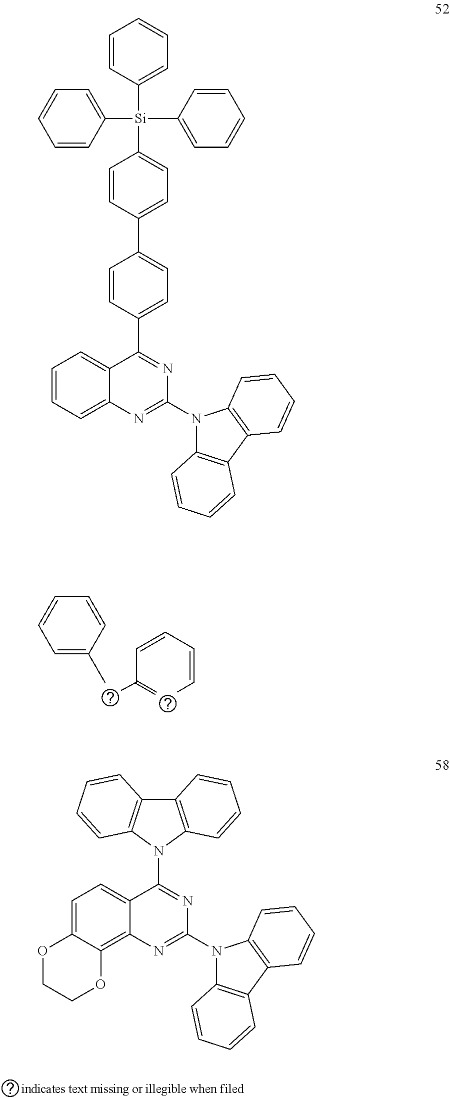

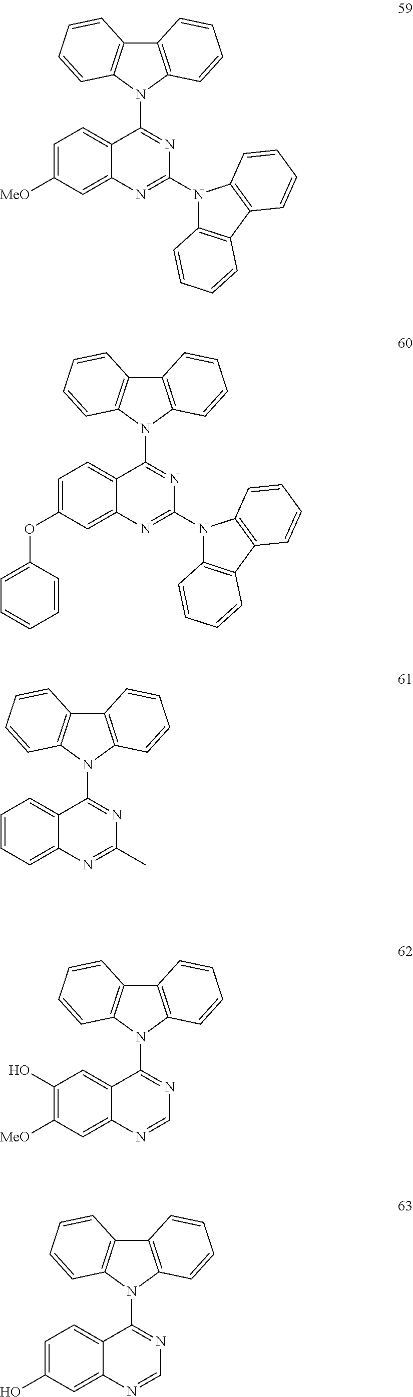

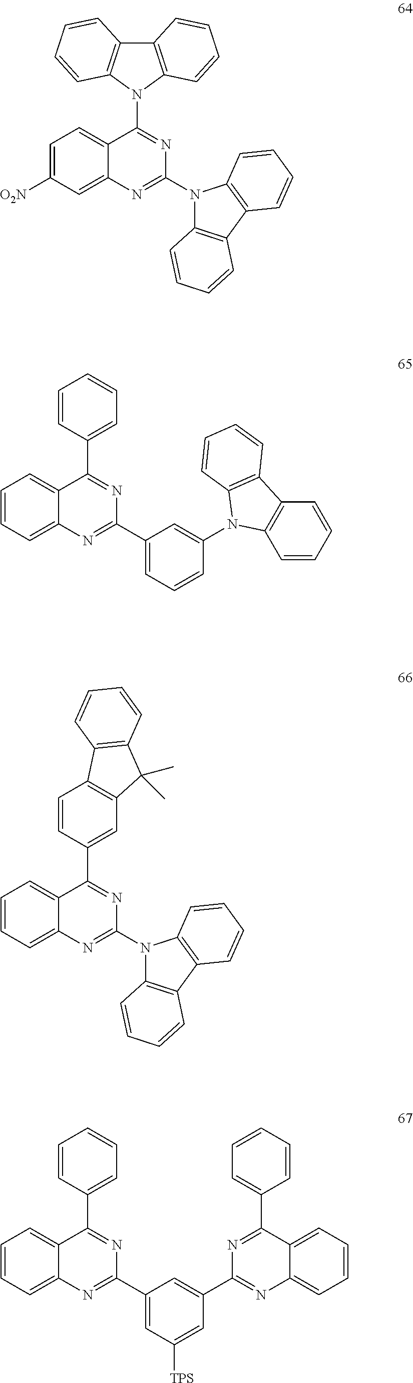

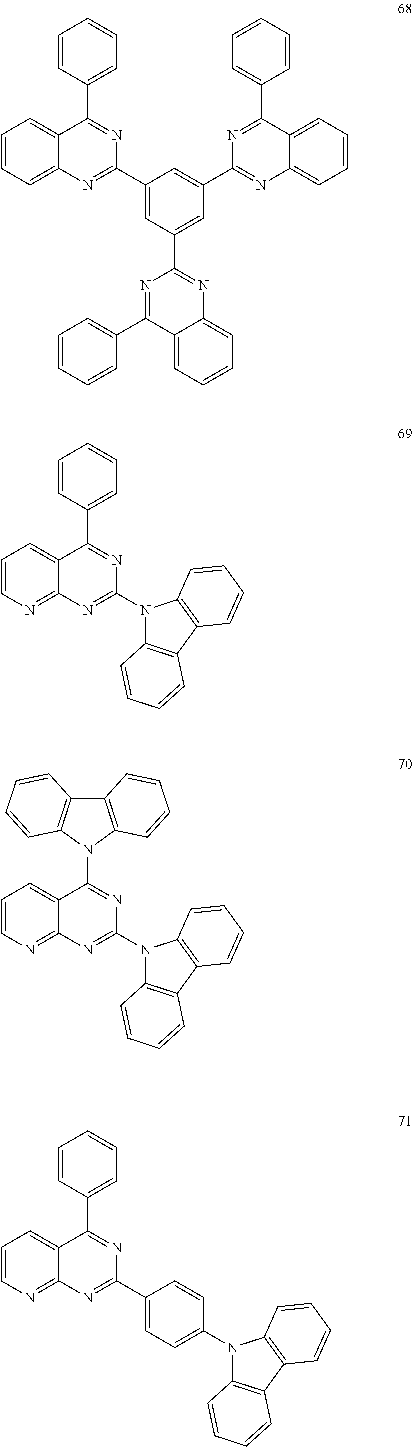

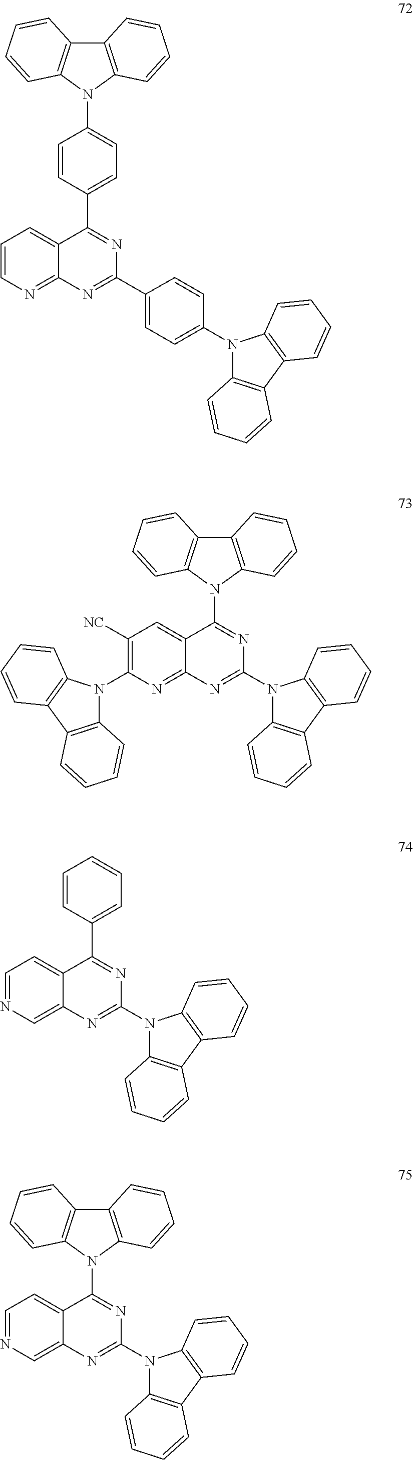

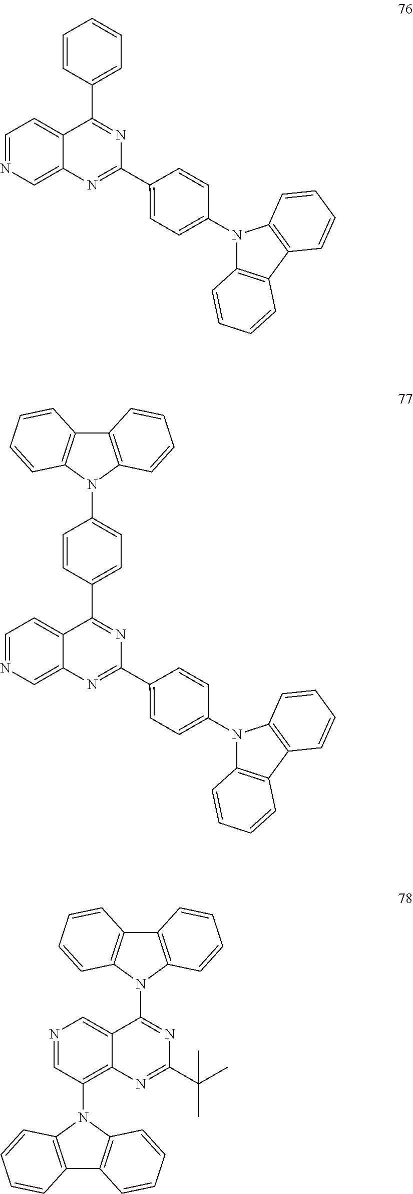

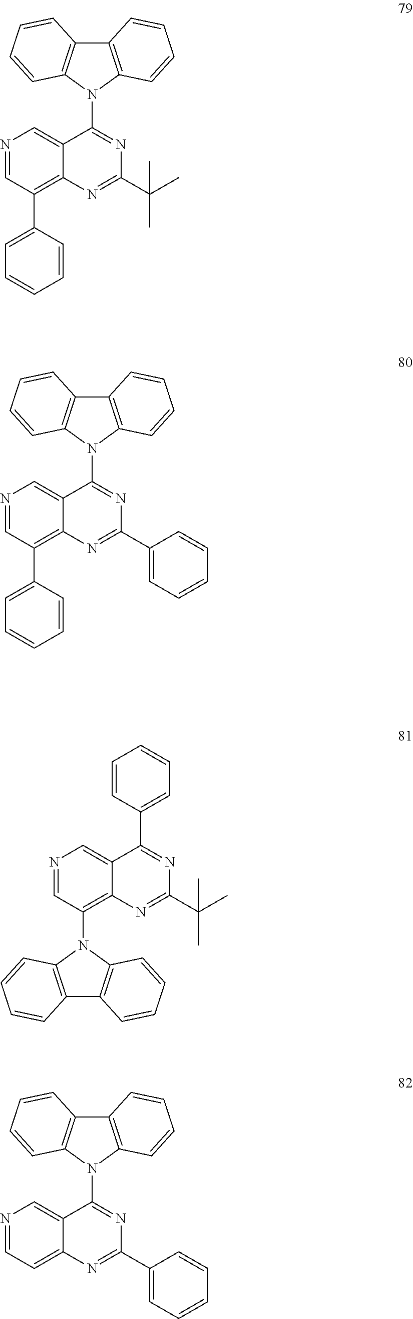

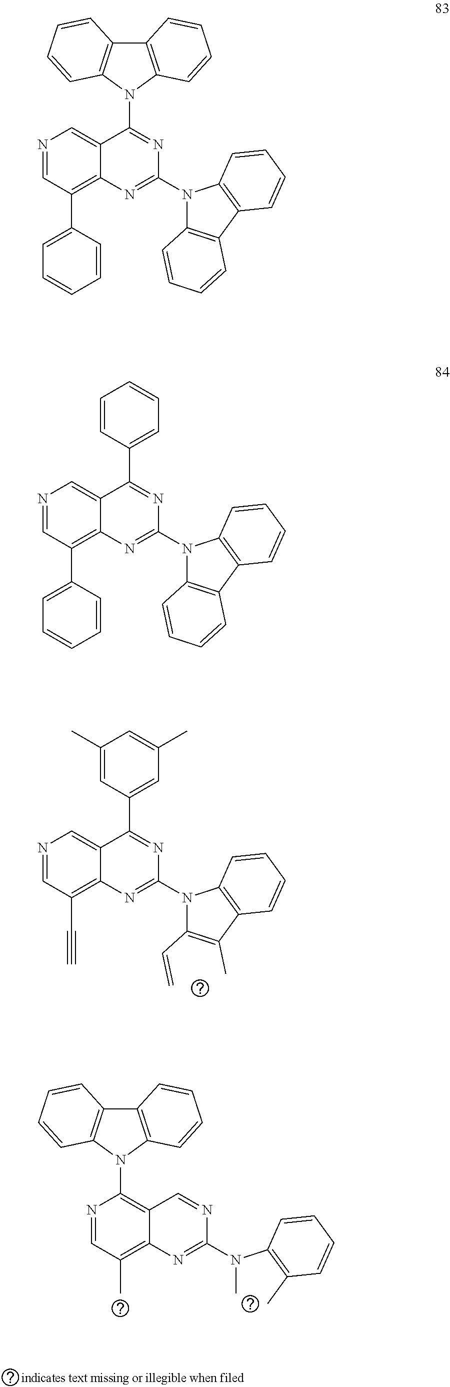

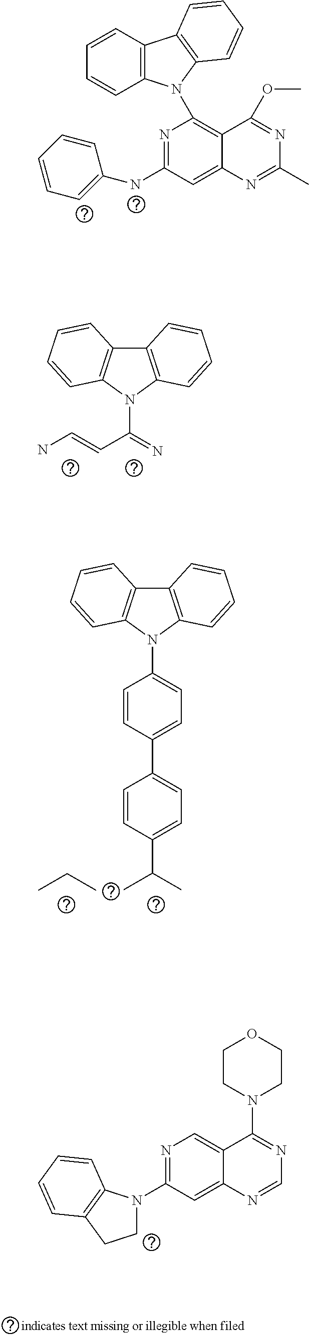

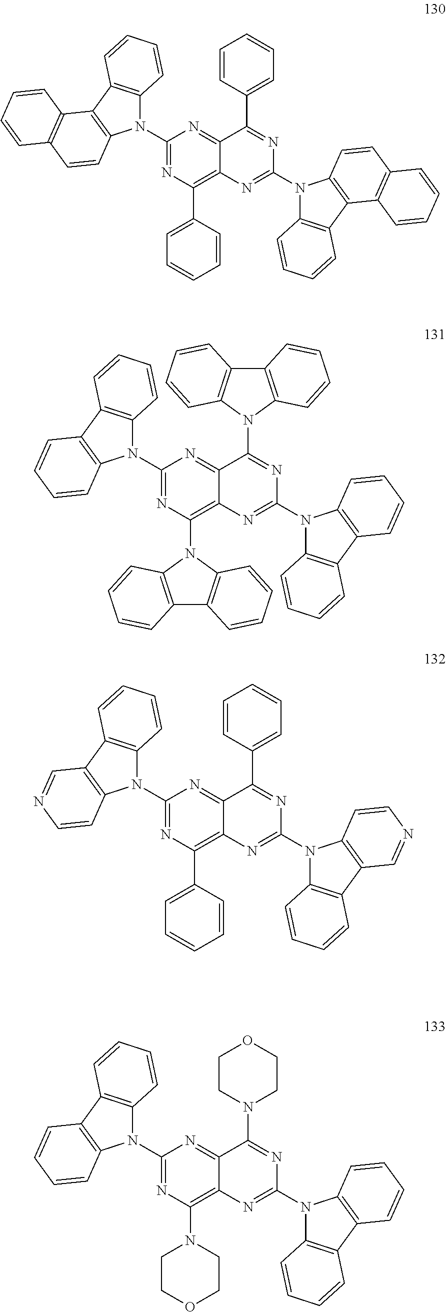

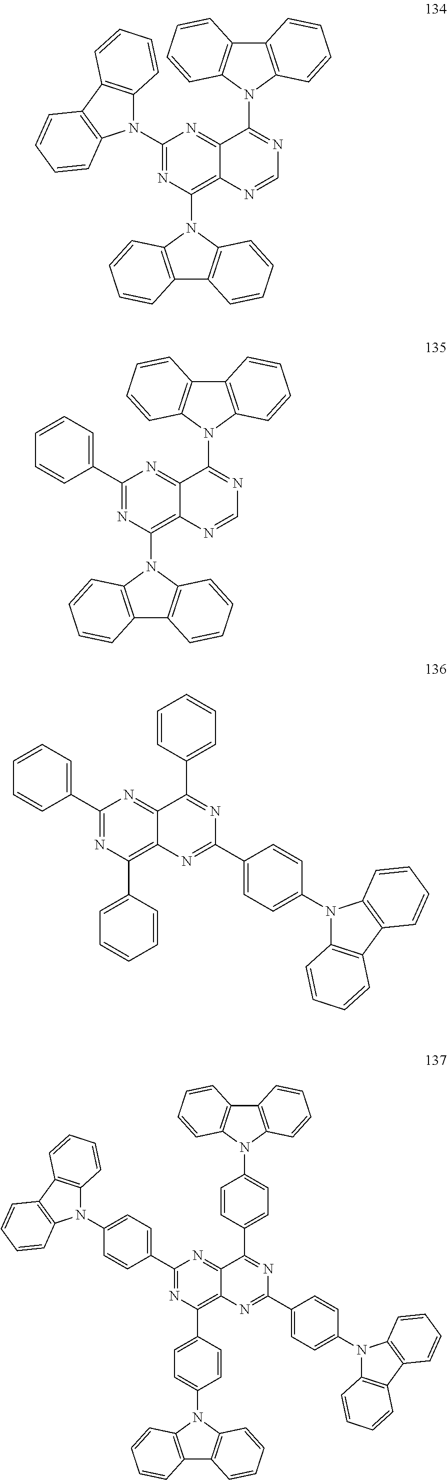

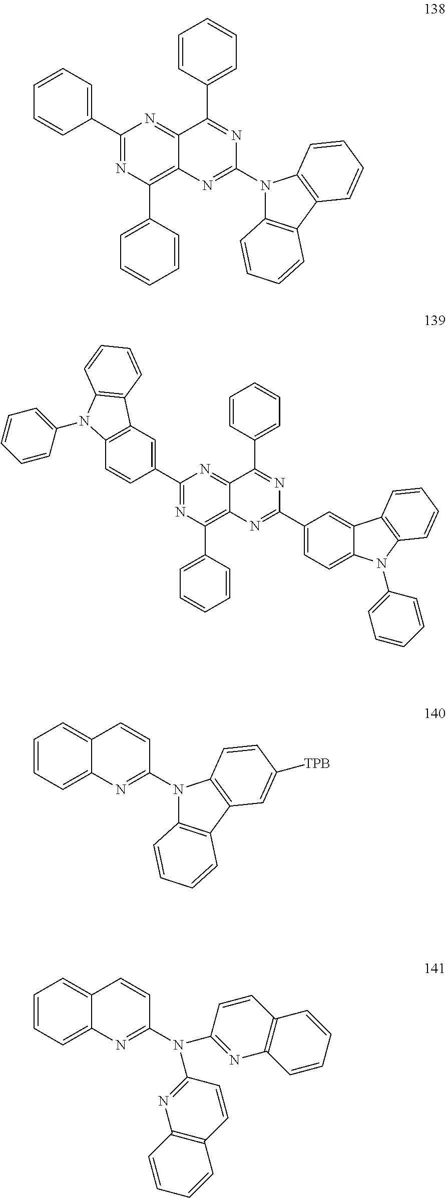

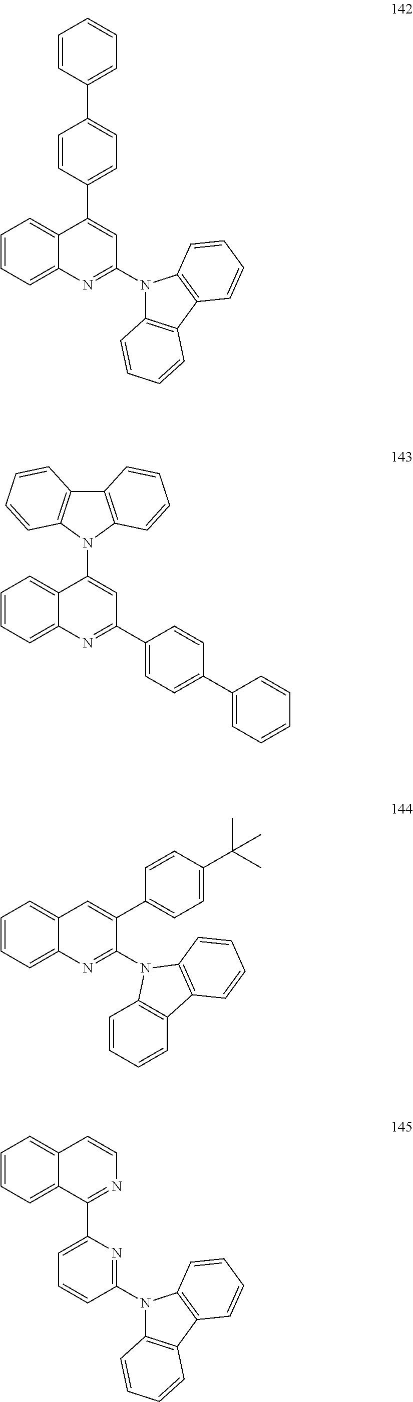

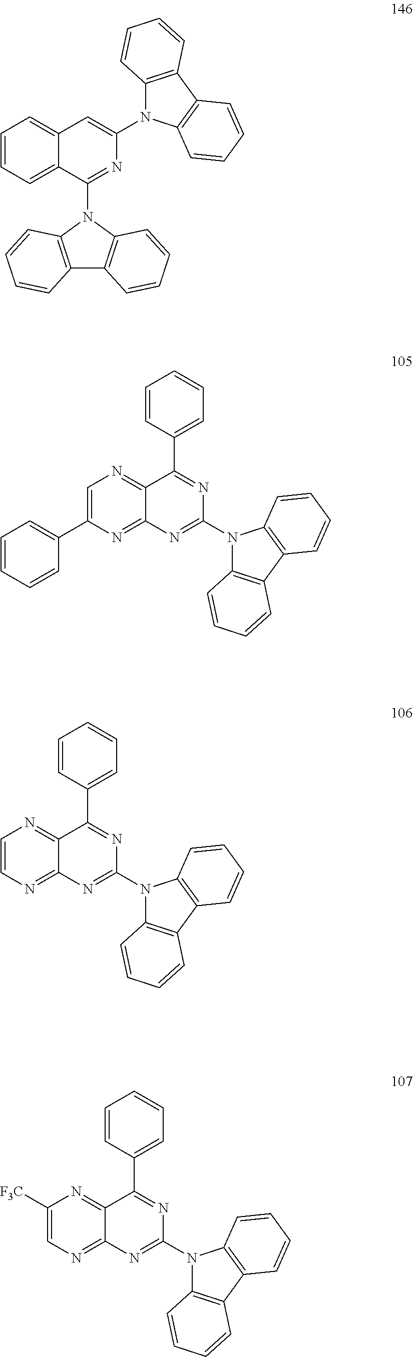

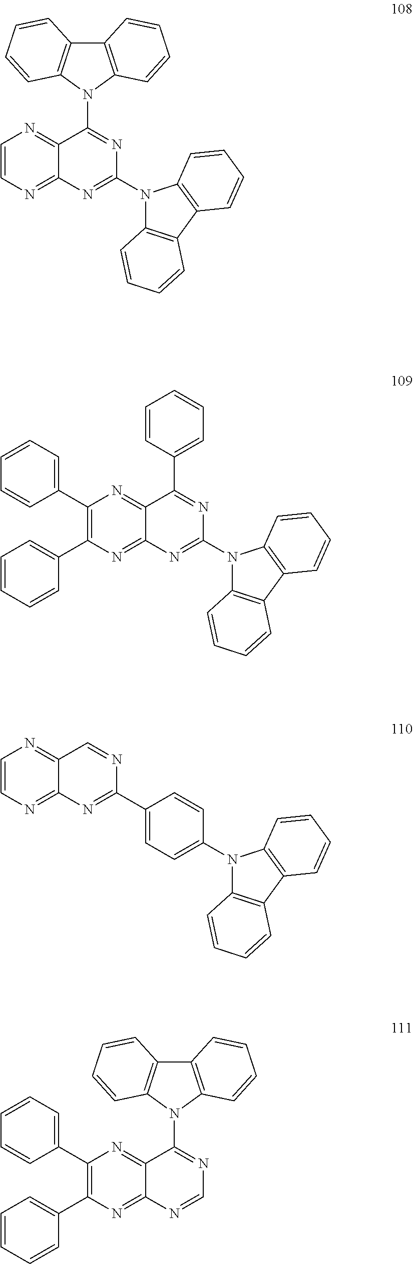

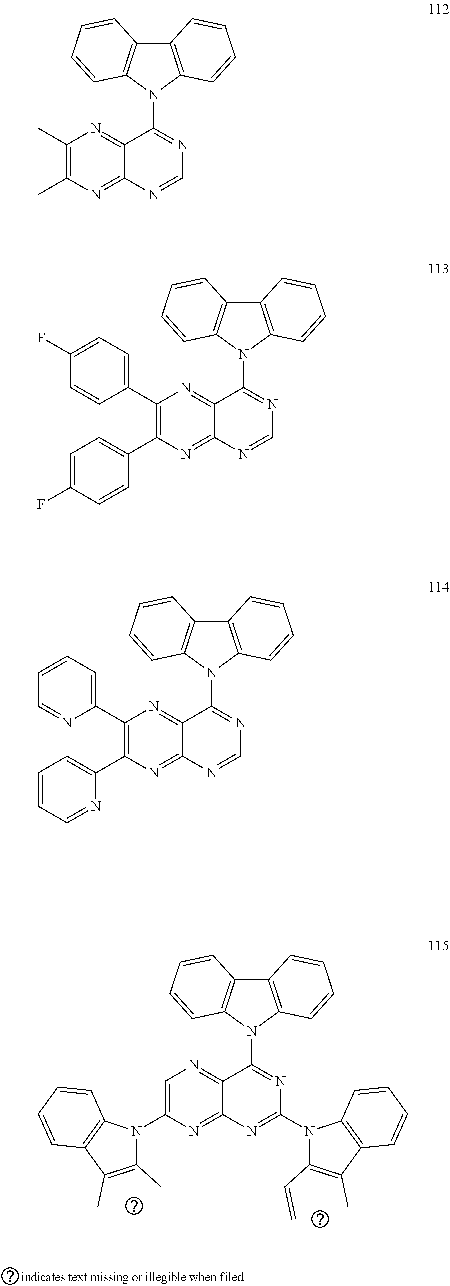

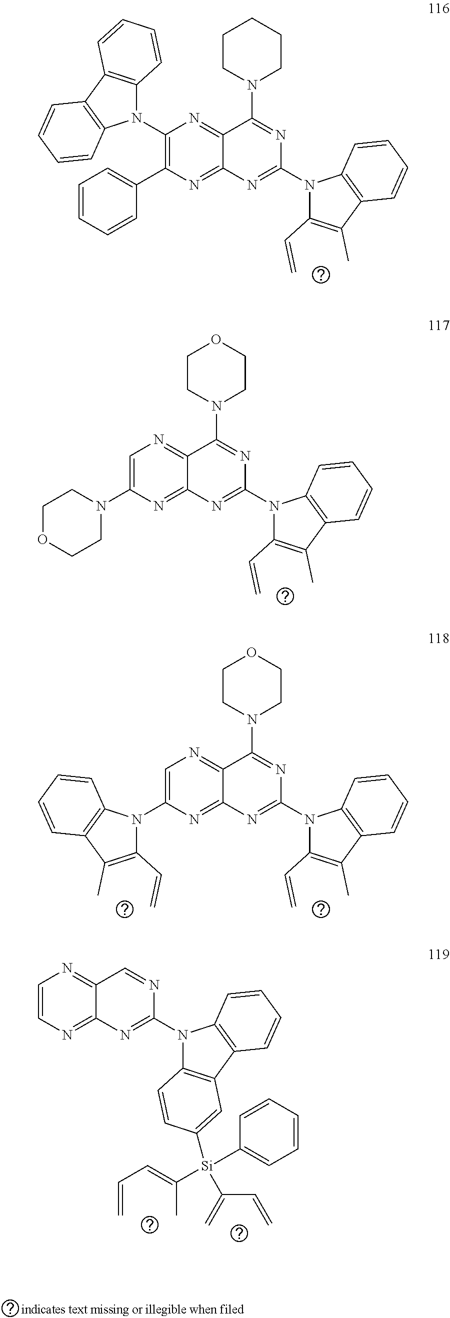

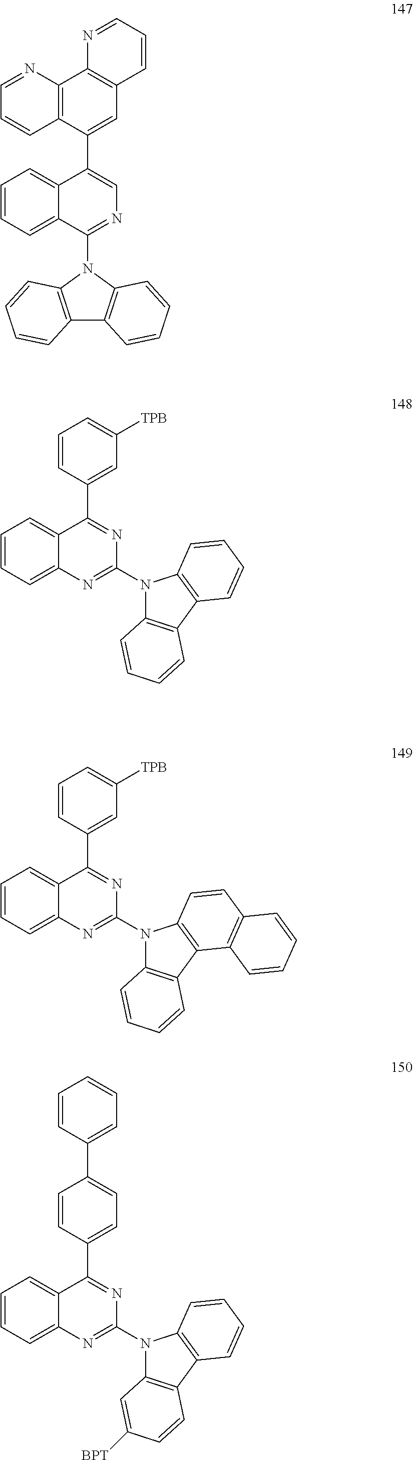

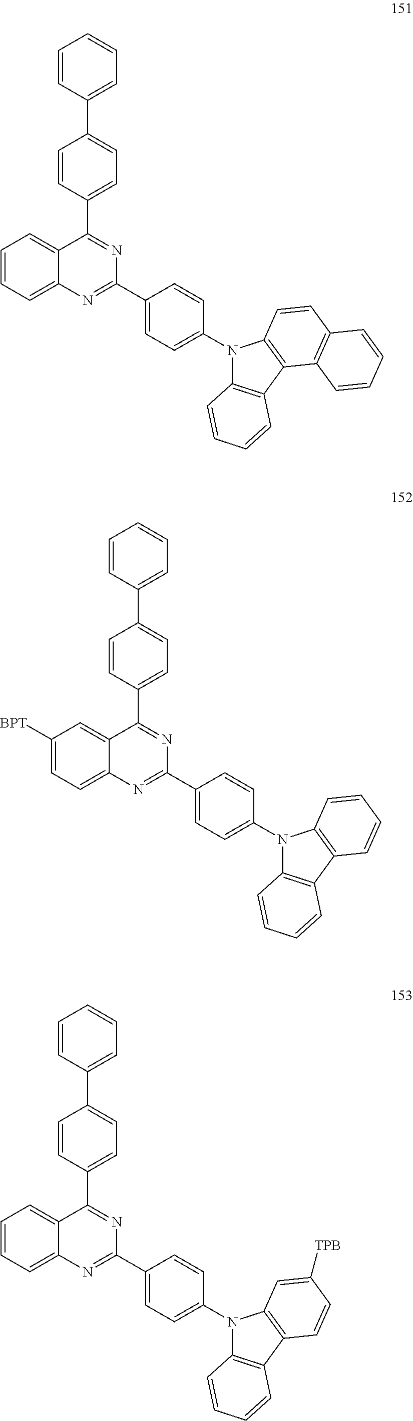

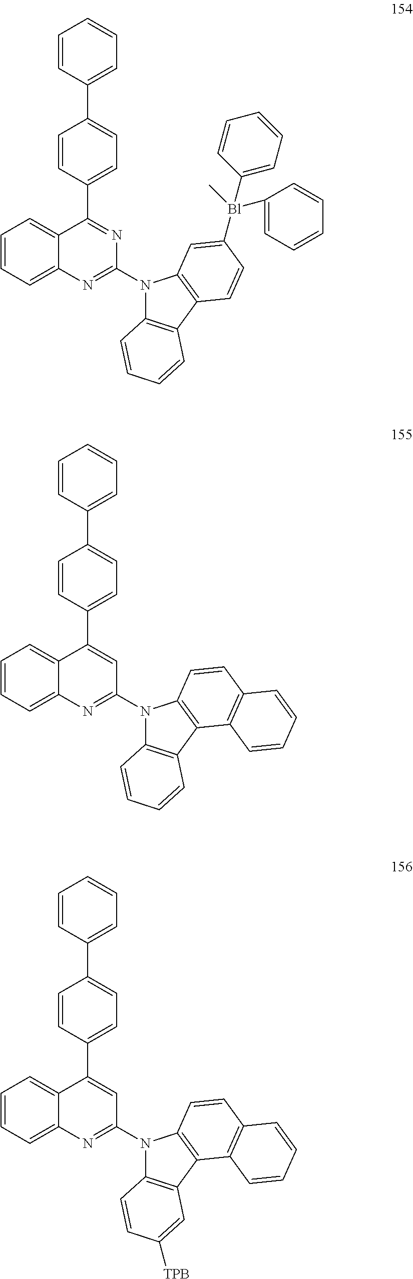

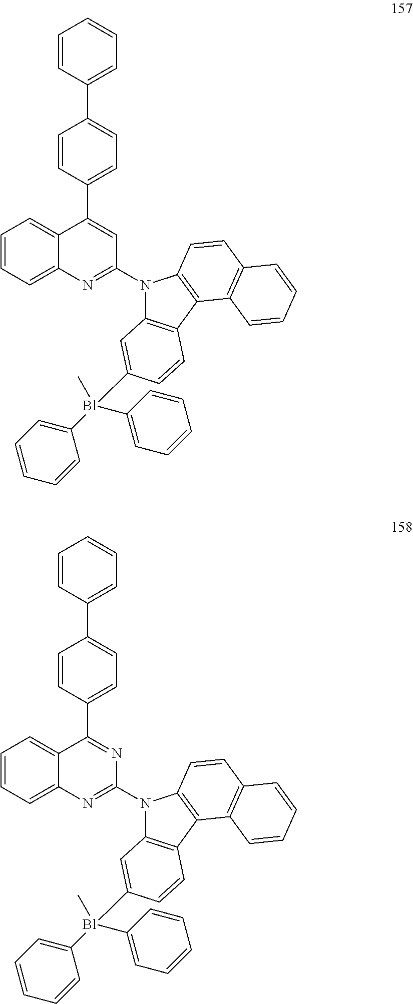

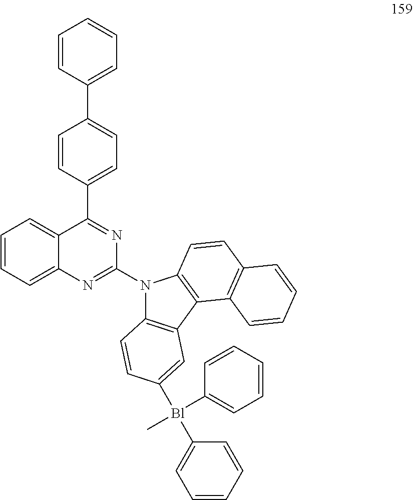

Provided is an organic electroluminescent device, which includes a first electrode; a second electrode; and one or more organic layer(s) interposed between the first electrode and the second electrode, wherein the organic layer includes one or more organic electroluminescent compound(s) represented by Chemical Formula 1. The organic electroluminescent compounds are used as host material of an electroluminescent layer.

In the organic electroluminescent device, the organic layer includes electroluminescent layer including one or more organic electroluminescent compound(s) represented by Chemical Formula 1 and one or more phosphorescent dopant(s). The electroluminescent dopant applied to the organic electroluminescent device is not specifically limited but may be exemplified as compounds of the following Chemical Formula 6.

M¹L¹⁰¹L¹⁰²L¹⁰³ Chemical Formula 6

M¹ is selected from the group consisting of metals of Group 7, Group 8, Group 9, Group 10, Group 11, Group 13, Group 14, Group 15 and Group 16, and ligands L¹⁰¹, L¹⁰² and L¹⁰³ are independently selected from following structures.

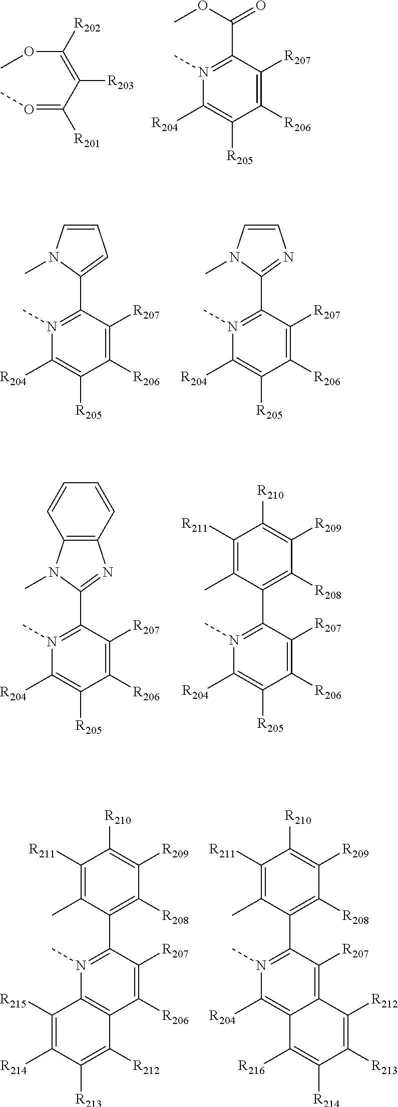

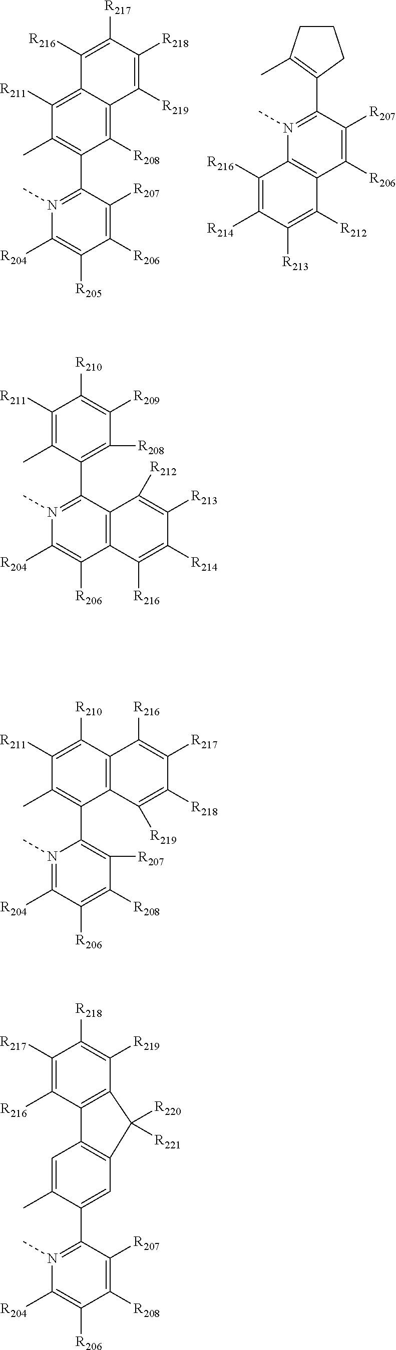

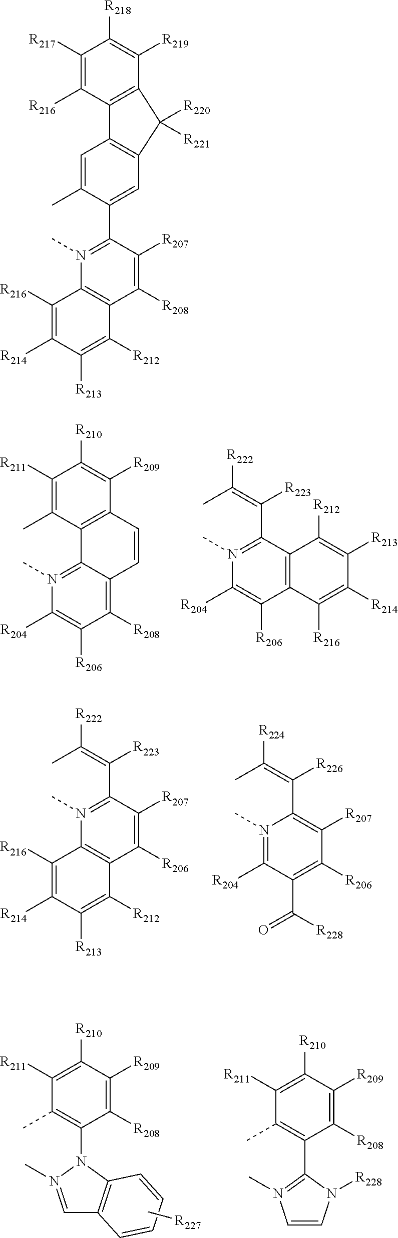

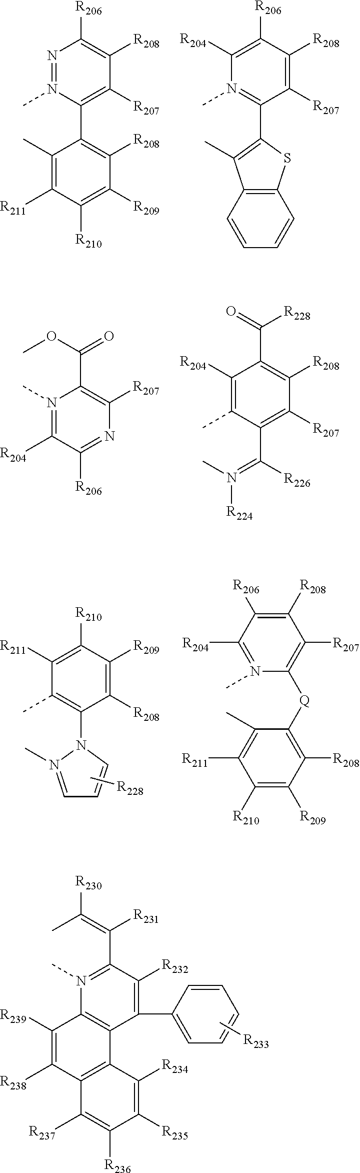

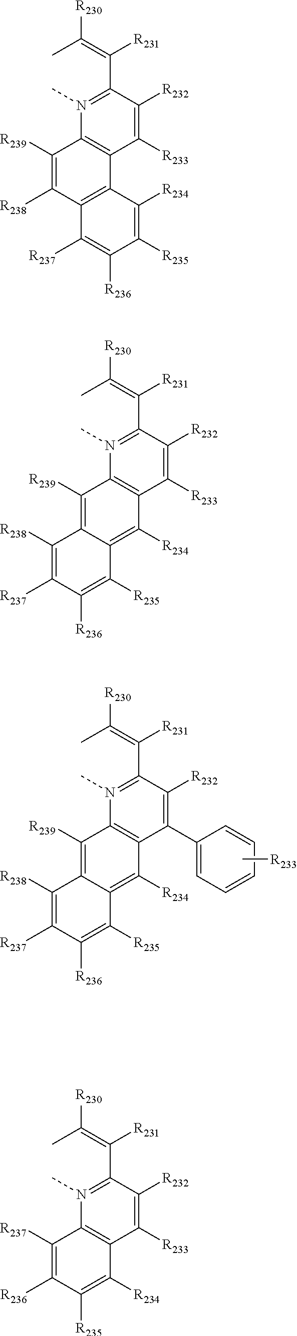

wherein
R₂₀₁ through R₂₀₃ independently represent hydrogen, deuterium, (C1-C30)alkyl with or without halogen substituent(s), (C6-C30)aryl with or without (C1-C30)alkyl substituent(s) or halogen;
R₂₀₄ through R₂₁₉ independently represent hydrogen, deuterium, (C1-C30)alkyl with or without substituent(s), (C1-C30)alkoxy with or without substituent(s), (C3-C30)cycloalkyl with or without substituent(s), (C2-C30)alkenyl with or without substituent(s), (C6-C30)aryl with or without substituent(s), mono or di-(C1-C30)alkylamino with or without substituent(s), mono or di-(C6-C30)arylamino with or without substituent(s), SF₅, tri(C1-C30)alkylsilyl with or without substituent(s), di(C1-C30)alkyl(C6-C30)arylsilyl with or without substituent(s), tri(C6-C30)arylsilyl with or without substituent(s), cyano or halogen;
R₂₂₀ through R₂₂₃ independently represent hydrogen, deuterium, (C1-C30)alkyl with or without halogen substituent(s) or (C6-C30)aryl with or without (C1-C30)alkyl substituent(s);
R₂₂₄ and R₂₂₅ independently represent hydrogen, deuterium, (C1-C30)alkyl with or without substituent(s), (C6-C30)aryl with or without substituent(s) or halogen, or R₂₂₄ and R₂₂₅ are linked via (C3-C12)alkylene or (C3-C12)alkenylene with or without a fused ring to form an alicylic ring or a mono- or polycyclic aromatic ring;
R₂₂₆ represents (C1-C30)alkyl with or without substituent(s), (C6-C30)aryl with or without substituent(s), (C5-C30)heteroaryl with or without substituent(s) or halogen;
R₂₂₇ through R₂₂₉ independently represent hydrogen, deuterium, (C1-C30)alkyl with or without substituent(s), (C6-C30)aryl or halogen with or without substituent(s);
R₂₃₀ and R₂₃₁ independently represent hydrogen, (C1-C20)alkyl with or without halogen substituent(s), (C6-C20)aryl, halogen, cyano, tri(C1-C20)alkylsilyl, di(C1-C20)alkyl(C6-C20)arylsilyl, tri(C6-C20)arylsilyl, (C1-C20)alkoxy, (C1-C20)alkylcarbonyl, (C6-C20)arylcarbonyl, di(C1-C20)alkylamino or di(C6-C20)arylamino, or R₂₃₀ and R₂₃₁ are linked via (C3-C12)alkylene or (C3-C12)alkenylene with or without a fused ring to form an alicylic ring or a mono- or polycyclic aromatic ring;
the alicylic ring or the mono- or polycyclic aromatic ring formed by being linked via alkyl of the R₂₃₀ and R₂₃₁, aryl or (C3-C12)alkylene or (C3-C12)alkenylene with or without a fused ring may be further substituted with one or more substituent(s) selected from the group consisting of (C1-C20)alkyl with or without halogen substituent(s), halogen, cyano, tri(C1-C20)alkylsilyl, di(C1-C20)alkyl(C6-C20)arylsilyl, tri(C6-C20)arylsilyl, (C1-C20)alkoxy, (C1-C20)alkylcarbonyl, (C6-C20)arylcarbonyl, di(C1-C20)alkylamino, di(C6-C20)arylamino, phenyl, naphthyl, anthryl, fluorenyl, or spirobifluorenyl, or may be further substituted with phenyl or fluorenyl, which is substituted with one or more substituent(s) selected from the group consisting of (C1-C20)alkyl with or without halogen substituent(s), halogen, cyano, tri(C1-C20)alkylsilyl, di(C1-C20)alkyl(C6-C20)arylsilyl, tri(C6-C20)arylsilyl, (C1-C20)alkoxy, (C1-C20)alkylcarbonyl, (C6-C20)arylcarbonyl, di(C1-C20)alkylamino, di(C6-C20)arylamino, phenyl, naphthyl, anthryl, fluorenyl, or spirobifluorenyl;
R₂₃₂ through R₂₃₉ independently represent hydrogen, (C1-C20)alkyl with or without halogen substituent(s), (C1-C20)alkoxy, (C3-C12)cycloalkyl, halogen, cyano, (C6-C20)aryl, (C4-C20)heteroaryl, tri(C1-C20)alkylsilyl, di(C1-C20)alkyl(C6-C20)arylsilyl or tri(C6-C20)arylsilyl;

Q represents

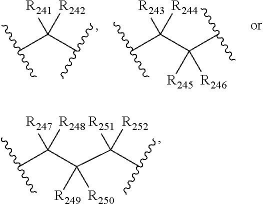

R₂₄₁ through R₂₅₂ independently represent hydrogen, (C1-C60)alkyl with or without halogen substituent(s), (C1-C30)alkoxy, halogen, (C6-C60)aryl, cyano, or (C5-C60)cycloalkyl, or R₂₄₁ through R₂₅₂ are linked to an adjacent substituent via alkylene or alkenylene to form a (C5-C7)spiro ring or a (C5-C9)fused ring, or are linked to R₂₀₇ or R₂₀₈ via alkylene or alkenylene to form a (C5-C7)fused ring.

The M¹ is selected from a group consisting of Ir, Pt, Pd, Rh, Re, Os, Tl, Pb, Bi, In, Sn, Sb, Te, Au and Ag, and the compounds of Chemical Formula 6 include the examples in KR Patent Application No. 10-2009-0037519, but is not limited thereto.

The organic electroluminescent device includes the organic electroluminescent compound of Chemical Formula 1 and includes one or more compound(s) selected from a group consisting of arylamine compounds and styrylarylamine compounds at the same time. The arylamine compounds and styrylarylamine compounds include the examples in KR Patent Application Nos. 10-2008-0123276, 10-2008-0107606 and 10-2008-0118428, but are not limited thereto.

The organic layer may further include one or more metal(s) selected from a group consisting of organic metals of Group 1, Group 2, 4th period and 5th period transition metals, lanthanide metals and d-transition elements or complex compound(s) as well as the organic electroluminescent compound of Chemical Formula 1. The organic layer may simultaneously include the electroluminescent layer and a charge generating layer.

Provided is a white light-emitting organic electroluminescent device wherein the organic layer includes one or more organic electroluminescent layer(s) emitting blue, red or green light at the same time as well as the organic electroluminescent compound.

### Advantageous Effects

The organic electroluminescent compound according to the present invention has good luminescence efficiency and excellent life property compared with the existing host material since it is used as host material of the organic electroluminescent material in the OLED. Accordingly, it may be used to manufacture an OLED having very superior operation life.

### MODE OF THE INVENTION

The organic electroluminescent compounds according to the present invention, processes for preparing the same, and luminescence properties of devices employing the same will be described in detail hereinafter based on the representative compound for easy understanding. However, the following examples are provided for illustrative purposes only and they are not intended to limit the scope of the present invention.

### Preparation Example 1

Preparation of Compound 24

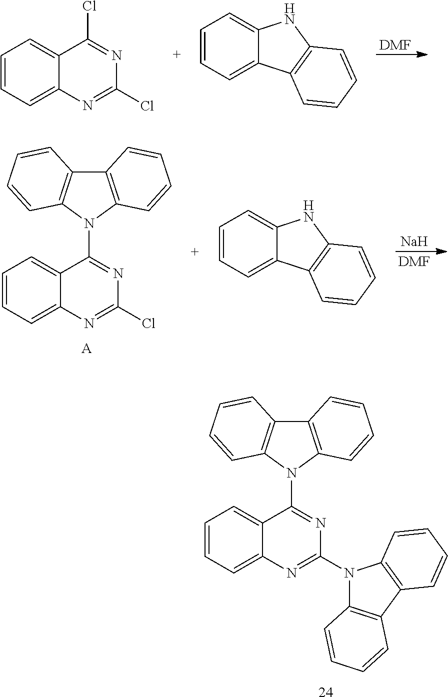

Preparation of Compound A

After dissolving 2,4-dichloroquinazolin (2 g, 10.0 mmol) in DMF (20 mL), carbazole (3.69 g,22.1 mmol) was added thereto and the mixture was stirred for 2 hours at room temperature. The reaction was completed by adding water to the reaction mixture. After extracting with ethyl acetate washing with 5% citric acid, and removing remaining moisture by using sodium sulfate, drying followed by column separation yielded Compound A (2.6 g, 7.88 mmol, 79%).

Preparation of Compound 24

After dissolving carbazole (1.97 g, 11.8 mmol) in DMF and adding solution, which was obtained by dissolving sodium hydride (605 mg, 15.8 mmol) in DMF, the mixture was stirred for 1 hour. Solution, which was obtained by dissolving Compound A (2.6 g, 7.88 mmol) in DMF, was added thereto and stirred at room temperature for 24 hours. The produced solid was filtered when the reaction was completed. Extracting with ethyl acetate followed by column separation yielded Compound 24 (2 g, 4.3 mmol, 71%).

### Preparation Example 2

Preparation of Compound 76

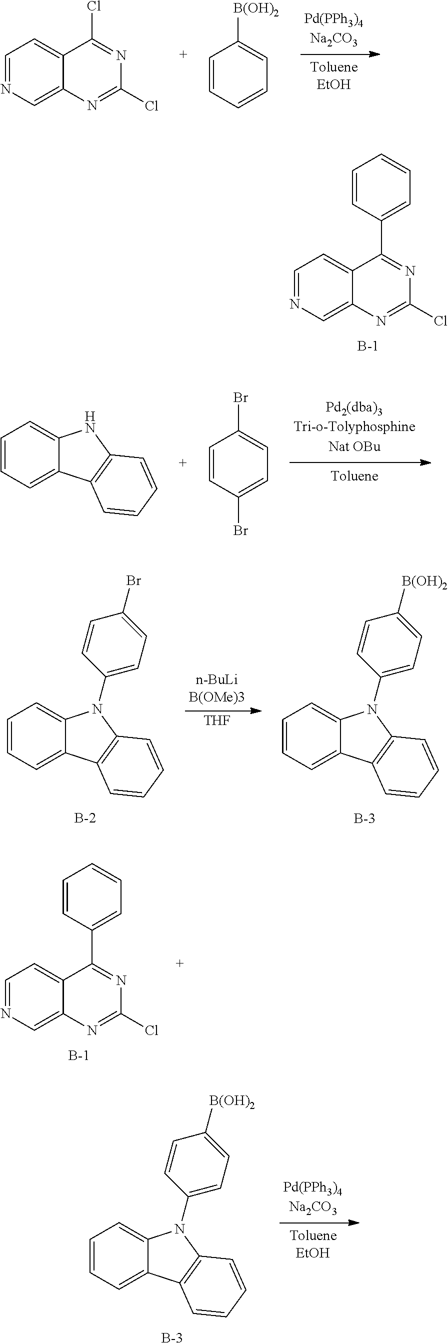

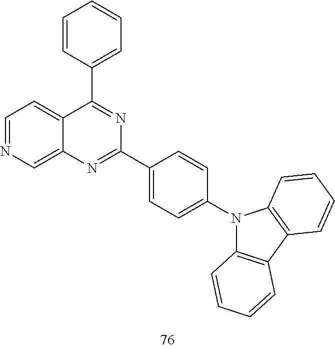

Preparation of Compound B-1

After 2,4-dichloropyrido[3,4-d]pyrimidin (5 g, 24.9 mmol), phenylboronic acid (3.6 g, 29.9 mmol), Pd(PPh₃)₄ ( 1/15 g, 0.996 mmol), sodium carbonate aqueous solution (87 mL), and ethanol (87 mL) were dissolved in toluene (180 mL), the mixture was stirred under reflux at 120° C. for 3 hours. When the reaction was completed, an organic layer was extracted with ethyl acetate and remaining moisture was removed by using magnesium sulfate. Drying followed by column separation yielded Compound B-1 (4 g, 16.5 mmol, 67%).

Preparation of Compound B-2

After carbazole (4 g, 23.9 mmol), 1,4-dibromo benzene (14.1 g, 59.8 mmol), Pd₂(dba)₃ (438 mg, 0.478 mmol), tri-ortho-tolylphosphine (436 mg, 1.43 mmol), and sodium t-butoxide (4.59 g, 47.8 mmol) were dissolved in toluene (120 mL), the mixture was stirred under reflux at 120° C. for 6 hours. When the reaction was completed, an organic layer was extracted with ethyl acetate and remaining moisture was removed by using magnesium sulfate. Drying followed by column separation yielded Compound B-2 (4 g, 12.4 mmol, 65%).

Preparation of Compound B-3

After Compound B-2 (4 g, 12.4 mmol) was dissolved in THF (40 mL), the mixture was cooled to −78° C. 10 monutes later, n-butyl lithium(2.5M in hexanee) (5.95 mL, 14.88 mmol) was slowly added into a flask and the mixture was stirred for 1 hour. Trimethyl borate (2.35 mL, 18.6 mmol) was slowly added into a flask and the mixture was stirred for 24 hours. When the reaction was completed, 1M HCl was added thereto. After extracting with ethyl acetate and removing remaining moisture by using magnesium sulfate, drying followed by column separation yielded Compound B-3 (2 g, 6.96 mmol, 57%).

Preparation of Compound 76

After Compound B-2 (1.4 g, 5.8 mmol), Compound B-3 (2 g, 6.96 mmol), Pd(PPh₃)₄ (268 mg, 0.232 mmol), sodium carbonate aqueous solution (8 mL), and ethanol (8 mL) were dissolved in toluene (20 mL), the mixture was stirred under reflux at 120° C. for 24 hours. When the reaction was completed, an organic layer was extracted with ethyl acetate and remaining moisture was removed by using magnesium sulfate. Drying followed by column separation yielded Compound 76 (1 g, 2.23 mmol, 40%).

### Preparation Example 3

Preparation of Compound 89

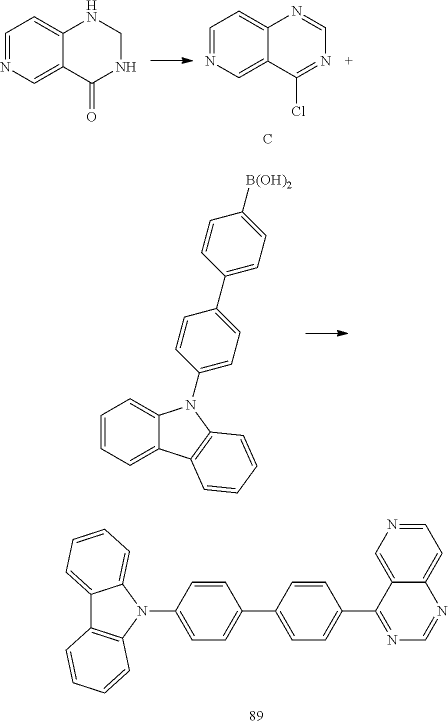

Preparation of Compound C

After 2,3-dihydropyrido[4,3-d]pyrimidin-4(1H) (5.0 g, 33.5 mmol) was dissolved in Phosphorus oxychloride (50 mL), the mixture was stirred under reflux at 100° C. for 5 hours. When the reaction was completed, a reactant was slowly added into ice water. After neutralizing with 5M NaOH and filtering dropping solid, Compound C (2.5 g, 15.0 mmol, 45%) was obtained via column separation.

Preparation of Compound 89

After 4-chloropyrido[4,3-d]pyrimidin (2.5 g, 15.0 mmol), 3-(9H-carbazole-9-one)biphenylboronic acid (8.1 g, 22.5 mmol), Pd(PPh₃)₄ 693 mg(0.60 mmol), sodium carbonate aqueous solution (52 mL), and ethanol (52 mL) were dissolved in toluene (100 mL), the mixture was stirred under reflux at 120° C. for 3 hours. After extracting with ethyl acetate when the reaction was completed, remaining moisture was removed by using magnesium sulfate. Drying followed by column separation yielded Compound 89 (3 g, 6.69 mmol, 50%)

### Preparation Example 4

Preparation of Compound 108

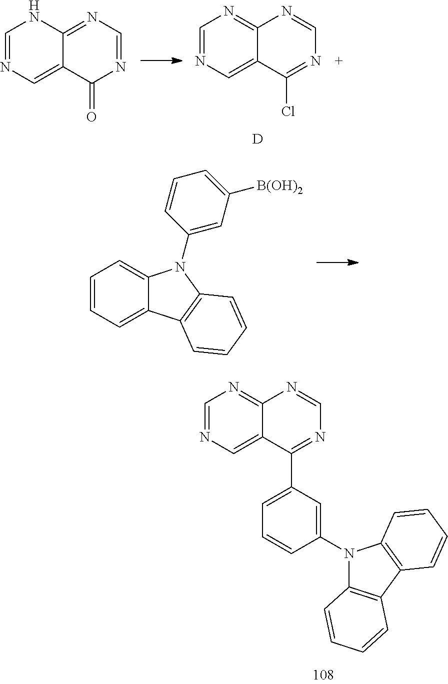

Preparation of Compound D

Pyrimido[4,5-d]pyrimidin-4(8H)-one (5.0 g, 33.7 mmol) was dissolved with Phosphorus oxychloride (50 mL) and stirred under reflux at 100° C. for 5 hours. When the reaction was completed, a reactant was slowly added into ice water. After neutralizing with 5M NaOH and filtering dropping solid, Compound D (2.2 g, 13.2 mmol, 40%) was obtained via column separation.

Preparation of Compound 108

After 4-chloropyrido[4,3-d]pyrimidin (2.2 g, 13.2 mmol), 3-(9H-carbazole-9-one)biphenylboronic acid (5.6 g, 19.8 mmol), Pd(PPh₃)₄ (610 mg, 0.52 mmol), sodium carbonate aqueous solution (46 mL), and ethanol (46 mL) were dissolved in toluene (100 mL), the mixture was stirred under reflux at 120° C. for 3 hours. After extracting with ethyl acetate when the reaction was completed, remaining moisture was removed by using magnesium sulfate. Drying followed by column separation yielded Compound 108 (3.4 g, 9.2 mmol, 70%).

### Preparation Example 5

Preparation of Compound 128

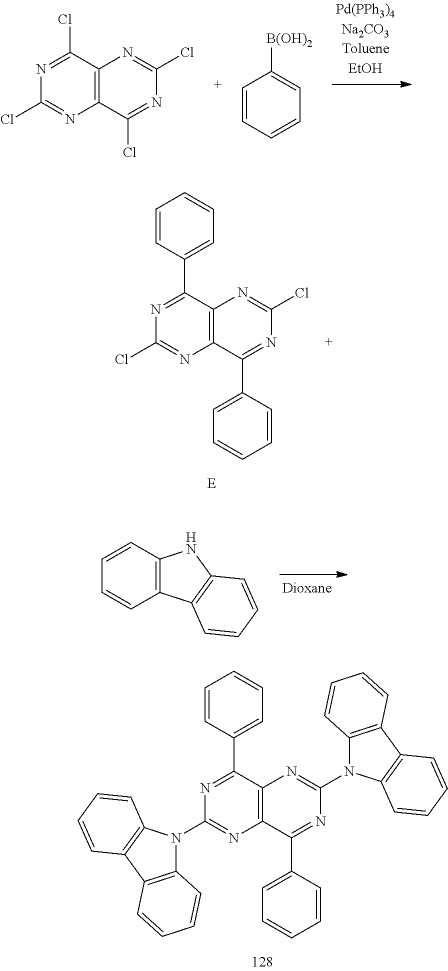

Preparation of Compound E

After perchloropyridido[5,4-d]pyrimidin (5 g, 18.5 mmol), phenylboronic acid (4.96 g, 40.7 mmol), Pd(PPh₃)₄ (856 mg, 0.74 mmol), sodium carbonate aqueous solution (65 mL), and ethanol (65 mL) were dissolved in toluene (200 mL), the mixture was stirred under reflux at 120° C. for 3 hours. After extracting with ethyl acetate when the reaction was completed, remaining moisture was removed by using magnesium sulfate. Drying followed by column separation yielded Compound E (5 g, 14.1 mmol, 77%).

Preparation of Compound 128

After Compound E (5 g, 14.1 mmol) and carbazole (5.2 g, 31.1 mmol) were dissolved in dioxane (50 mL), the mixture was stirred under reflux at 100° C. for 1 hour. After extracting with ethyl acetate when the reaction was completed, remaining moisture was removed by using magnesium sulfate. Drying followed by column separation yielded Compound 128 (7 g, 16.2 mmol, 80%).

### Preparation Example 6

Preparation of Compound 150

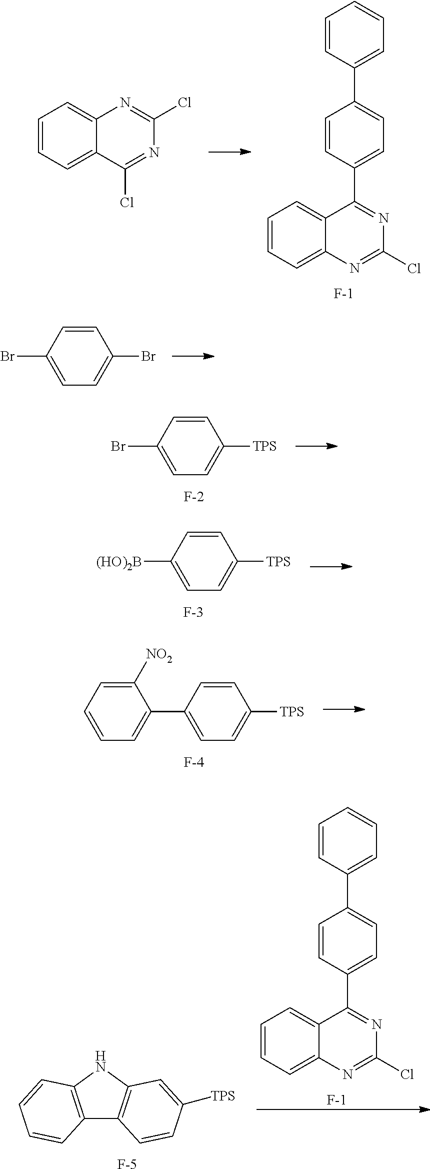

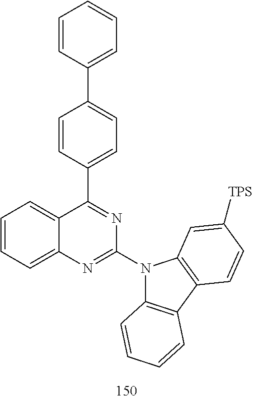

Preparation of Compound F-1

After 2,4-Dichloroquinazolin (15 g, 75.36 mmol), 4-biphenylboronic acid (16.4 g, 82.89 mmol), Pd(PPh₃)₄ (3.76 mmol), 2M-Na₂CO₃ (110 mL) and ethanol (50 mL) were dissolved in toluene (300 mL), the mixture was stirred under reflux at 100° C. for 3 hours. Upon completion of the reaction, the mixture was cooled at room temperature. After extracting with ethyl acetate and washing with distilled water, remaining moisture was removed by using magnesium sulfate. Drying followed by column separation yielded Compound F-1 (12 g, 37.88 mmol, 50.26%).

Preparation of Compound F-2

1,4-dibromobenzene (15 g, 63.58 mmol) was dissolved in tetrahydrofuran (300 mL) and n-butyl lithium (26.7 mL, 2.5M in hexanee, 66.76 mmol) was slowly added thereto at −78° C. After the mixture was stirred for 1 hour, solution, in which chlorotriphenylsilane (20.6 mL) was dissolved in tetrahydrofuran (70 mL), was added thereto and stirred at room temperature for 12 hours. Upon completion of the reaction, the produced white solid was removed by filtering and filtrate was extracted with ethyl acetate. After washing with distilled water, remaining moisture was removed by using magnesium sulfate and distillation under reduced pressure was performed. Compound F-2 (17.5 g, 42.12 mmol, 66.25%) was obtained by recrystallization with ethyl acetate and methanol.

Preparation of Compound F-3

Compound F-2 (20 g, 48.14 mmol) was dissolved in tetrahydrofuran (500 mL) and n-butyl lithium (23.1 mL, 2.5M in Hexane, 57.5 mmol) was slowly added thereto at −78° C. After the mixture was stirred for 1 hour, trimethylborate (8.5 mL, 77.03 mmol) was added thereto and stirred at room temperature for 12 hours. After extracting with ethyl acetate and washing with distilled water, Compound F-3 (14 g, 36.81 mmol, 76.46%) was obtained by recrystallization with ethyl acetate and n-hexane.

Preparation of Compound F-4

After 2-bromonitrobenzene (5 g, 24.75 mmol), Compound F-3 (10.35 g, 27.22 mmol), Pd(PPh₃)₄ (1.4 g, 1.23 mmol), 2M-K₂CO₃ (35 mL), and ethanol (50 mL) were dissolved in toluene (100 mL), the mixture was stirred under reflux at 100° C. for 12 hours. Upon completion of the reaction, the mixture was cooled at room temperature. After washing with distilled water and extracting an organic layer with ethyl acetate, remaining moisture was removed by using magnesium sulfate. Drying followed by recrystallization with ethyl acetate and methanol yielded Compound F-4 (11 g, 24.03 mmol, 97.12%).

Preparation of Compound F-5

After Compound F-4 (11 g, 24.03 mmol) was dissolved in 1,2-Dichlorobenzene (100 mL) and triethylphosphite (200 mL) was added thereto, the mixture was stirred at 150° C. for 15 hours. After distillation under reduced pressure, the produced solid was dissolved in chloroform and Compound F-5 (8 g, 18.81 mmol, 78.40%) was obtained by silica filtering.

Preparation of Compound 150

After Compound F-5 (4.8 g, 11.36 mmol) was dissolved in dimethylformamide (100 mL), the mixture was added into solution, in which NaH (0.56 g, 14.20 mmol) was dissolved in DMF (25 mL). the resultant mixture was stirred at room temperature for 1 hour. Subsequently, solution, in which Compound F-1 (3 g, 9.47 mmol) was dissolved in dimethylformamide (50 mL), was added thereto and the mixture was stirred at room temperature for 12 hours. After washing with distilled water and extracting with ethyl acetate, distillation under reduced pressure was performed. After the product was dissolved in chloroform and silica filtering was performed. Compound 150 (2.8 g, 5.46 mmol, 57.68%) was obtained by recrystallization with methanol, ethyl acetate and dimethylformamide.

### Preparation Example 7

Preparation of Compound 154

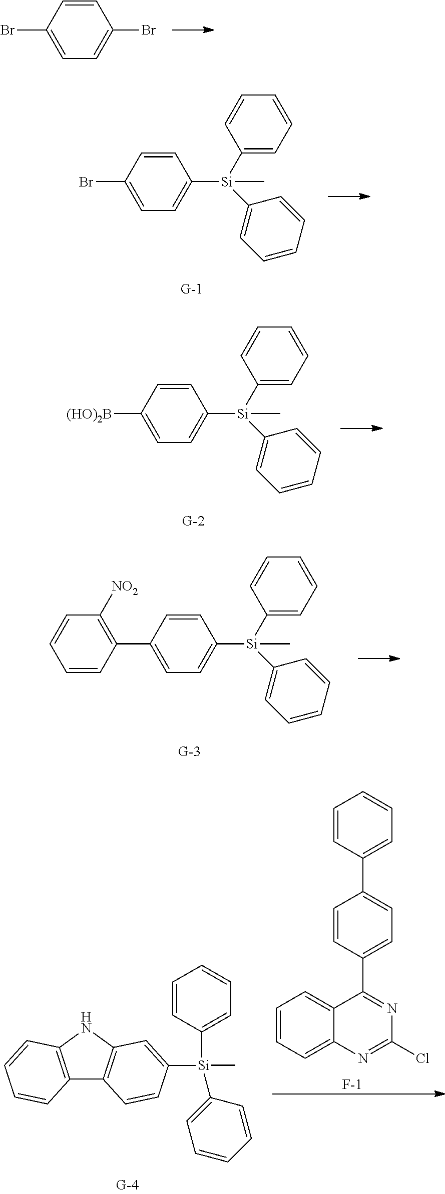

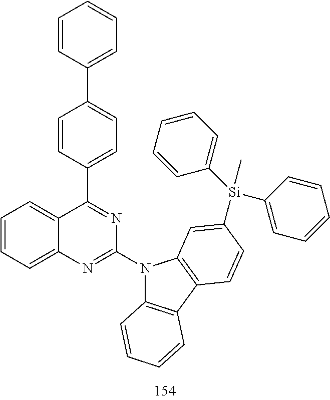

Preparation of Compound G-1

After 1,4-dibromobenzene (20 g, 84.78 mmol) was dissolved in tetrahydrofuran (550 mL) and n-butyl lithium (37.3 mL, 2.5M in hexanee, 93.25 mmol) was slowly added thereto at −78° C., the mixture was stirred for 1 hour. Solution, in which diphenylmethylsilylchloride (21.3 mL) was dissolved in tetrahydrofuran (50 mL), was added thereto and the mixture was stirred at room temperature for 12 hours. After extracting with ethyl acetate when the reaction was completed and washing with distilled water, remaining moisture was removed by using magnesium sulfate. Drying followed by distillation under reduced pressured was performed. After dissolving the product in chloroform, Compound G-1 (16 g, 45.28 mmol, 53.9%) was obtained by silica filtering.

Preparation of Compound G-2

After Compound G-1 (16 g, 45.28 mmol) was dissolved in tetrahydrofuran (200 mL) and n-butyl lithium (21.7 mL, 2.5M in Hexane, 54.34 mmol) was slowly added thereto at −78° C., the mixture was stirred for 1 hour. Trimethylborate (7.57 mL, 67.92 mmol) was added thereto and stirred at room temperature for 12 hours. After extracting with ethyl acetate and washing with distilled water, Compound G-2 (12 g, 37.70 mmol, 83.27%) was obtained by recrystallization with ethyl acetate and n-hexane.

Preparation of Compound G-3

After 2-bromonitrobenzene (7 g, 34.65 mmol), Compound G-2 (12.13 g, 38.11 mmol), Pd(PPh₃)₄ (2.0 g, 1.73 mmol), 2M-K₂CO₃ (40 mL), and ethanol (50 mL) were dissolved in toluene (120 mL), the mixture was stirred at 100° C. for 12 hours. Upon completion of the reaction, the mixture was cooled at room temperature. After washing with distilled water and extracting an organic layer with ethyl acetate, remaining moisture was removed by using magnesium sulfate. After drying and distillation under reduced pressure were performed, the obtained compound was dissolved in methylene chloride and silica filtering was performed. Compound G-3 (13 g, 32.86 mmol, 94.85%) was obtained by distillation under reduced pressure.

Preparation of Compound G-4

After Compound G-3 (13 g, 32.86 mmol) was dissolved in 1,2-Dichlorobenzene (150 mL) and triethylphosphite (200 mL) was added thereto, the mixture was stirred at 150° C. for 15 hours. After distillation under reduced pressure, the produced solid was dissolved in chloroform and Compound G-4 (10.5 g, 28.88 mmol, 87.80%) was obtained by silica filtering.

Preparation of Compound 154

After Compound G-4 (5.04 g, 13.88 mmol) was dissolved in dimethylformamide (30 mL), the mixture was added into solution, in which NaH (0.75 g, 18.95 mmol) was dissolved in DMF (30 mL). The resultant mixture was stirred at room temperature for 1 hour. After solution, in which Compound F-1 (4 g, 12.62 mmol) was dissolved in dimethylformamide (140 mL), was added thereto, the mixture was stirred at room temperature for 12 hours. After washing with distilled water and extracting with ethyl acetate, distillation under reduced pressure were performed. After dissolving the product in chloroform and performing silica filtering, Compound 154 (3.6 g, 5.59 mmol, 44.37%) was obtained by recrystallization with methanol, ethyl acetate and dimethylformamide.

### Preparation Example 8

Preparation of Compound 156

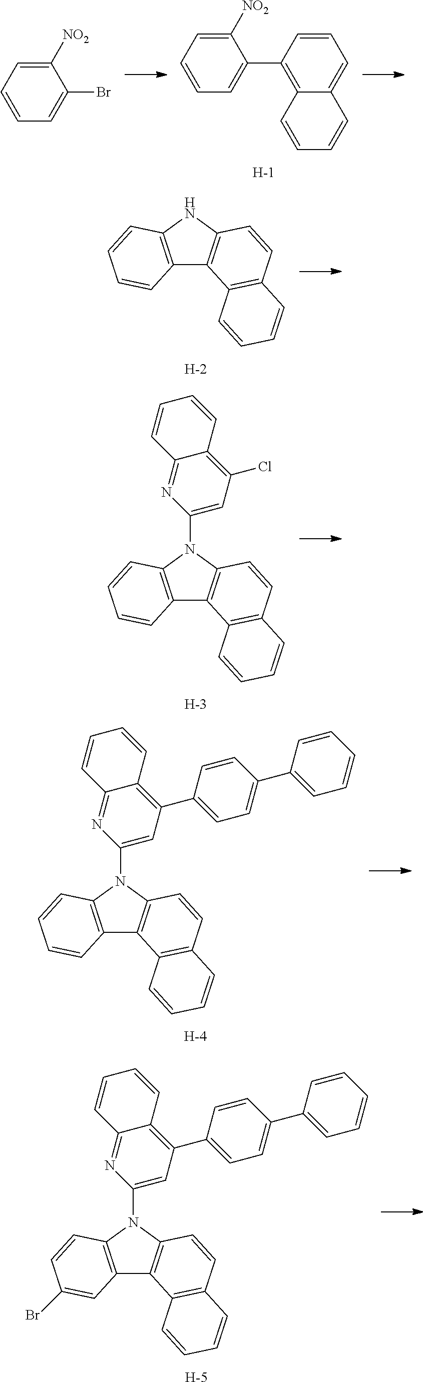

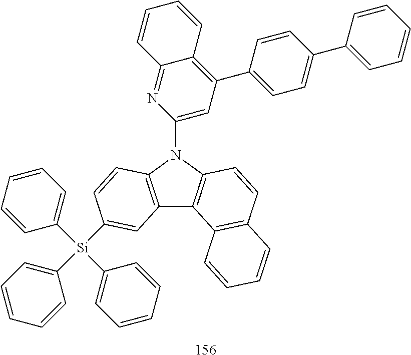

Preparation of Compound H-1

After 2-bromonitrobenzene (40 g, 0.198 mol), 1-naphthalene boronic acid (37.46 g, 0.217 mol), Pd(PPh₃)₄ (6.86 g, 0.005 mol), 2M-K₂CO₃ (200 mL) and ethanol (100 mL) were dissolved in toluene (300 mL), the mixture was stirred under reflux at 100° C. for 4 hours. Upon completion of the reaction, the mixture was cooled at room temperature. After washing with distilled water and extracting an organic layer with ethyl acetate, remaining moisture was removed by using magnesium sulfate. After drying and distillation under reduced pressure were performed, the obtained compound was dissolved in methylene chloride and silica filtering was performed. Compound H-1 (45 g, 0.180 mol, 91.17%) was obtained by distillation under reduced pressure.

Preparation of Compound H-2

After Compound H-1 (45 g, 0.180 mol) was dissolved in 1,2-Dichlorobenzene (200 mL) and triethylphosphite (400 mL) was added, the mixture was stirred at 150° C. for 12 hours. After the mixture was cooled at room temperature and distillation under reduced pressure was performed, Compound H-2 (32 g, 0.147 mol, 81.82%) was obtained by column separation.

Preparation of Compound H-3

After 2,4-Dichloroquinoline (15 g, 75.7 mmol), CuI (28.8 g, 151.4 mmol), Cs₂CO₃ (73 g, 227.2 mmol), trans-1,2-diaminocyclohexane (2.713 mL, 22.72 mmol), and 1,2-Dichlorobenzene (500 mL) was added into Compound H-2 (24.6 g, 113.6 mmol), the mixture was stirred under reflux at 180° C. for 12 hours. Upon completion of the reaction, the mixture was cooled at room temperature. After washing with distilled water and extracting an organic layer with ethyl acetate, remaining moisture was removed by using magnesium sulfate. After drying and distillation under reduced pressure were performed, Compound H-3 (20 g, 52.79 mmol, 70.38%) was obtained by column separation.

Preparation of Compound H-4

Compound H-3 (20 g, 52.79 mmol), 4-biphenylboronic acid (31 g, 158.3 mmol), Pd(OAc)₂ (1.18 g, 5.27 mmol), P(t-butyl)₃ (5.2 mL, 50% in toluene, 10.5 mmol), and K₃PO₄ (33.6 g, 158.3 mmol) were added to 1,4-dioxane (600 mL) and the mixture was stirred under reflux at 100° C. for 5 hours. Upon completion of the reaction, the mixture was cooled at room temperature. After washing with distilled water and extracting an organic layer with ethyl acetate, remaining moisture was removed by using magnesium sulfate. After drying and distillation under reduced pressure were performed, Compound H-4 (15 g, 30.20 mmol, 57.21%) was obtained by column separation.

Preparation of Compound H-5

After Compound H-4 (10 g, 20.13 mmol) was dissolved in tetrahydrofuran (300 mL) and NBS (3.94 g, 22.15 mmol) was added thereto at room temperature, the mixture was stirred at room temperature for 12 hours. Upon completion of the reaction, the mixture was cooled at room temperature. After washing with distilled water and extracting an organic layer with ethyl acetate, remaining moisture was removed by using magnesium sulfate. After drying and distillation under reduced pressure were performed, Compound H-5 (9.5 g, 16.50 mmol, 82.53%) was obtained by column separation.

Preparation of Compound 156

After Compound H-5 (10 g, 17.36 mmol) was dissolved in tetrahydrofuran (200 mL) and n-butyl lithium (13.89 mL, 2.5M in Hexane, 34.73 mmol) was slowly added thereto at −78° C., the mixture was stirred for 1 hour. After triphenylchlorosilane (10.2 g) was dissolved in tetrahydrofuran (200 mL), the mixture was stirred at room temperature for 12 hours. Upon completion of the reaction, distilled water was added thereto. After extracting with ethyl acetate and washing with distilled water, drying with magnesium sulfate and distillation under reduced pressure were performed. Compound 156 (5 g, 6.62 mmol, 38.14%) was obtained by column saparation.

In addition, diverse compounds represented by Compounds 2 to 5 may be may be combined by introducing diverse substituents according to known combination methods with one or more selected from the group consisting of 2,4-Dichloropyrido[2,3-d]pyrimidine(OChem Incorporation), 2-Chloro-pyrido[3,2-d]pyrimidine(Anichem LLC), pyrido[4,3-d]pyrimidine-4(3H)-one(Aces Pharma, Inc.), 2-Chloro-6,7-dimethyl-pteridine(International Laboratory Limited), 2-Chloropteridine (Princeton BioMolecular Research, Inc.), 3-Chloroquinoline (Texas Biochemicals Inc.), 2,4-Dichloroquinoline (Shanghai PI Chemicals Ltd), 2,3-Dichloroquinoline (Aces Pharma, Inc.), 1-Chloroisoquinoline (Alfa Aesar, China Ltd.), 1,3-Dichloroisoquinoline (Aalen Chemical Co. Ltd.), 1,4-Dichloroisoquinoline (Bepharm Ltd) as a starting material.

Organic electroluminescent Compounds 1 to 159 were prepared according to Preparation Examples 1 to 8 and Table 1 shows ¹H NMR and MS/FAB of the prepared organic electroluminescent compounds.

TABLE 1

| | | MS/FAB | |
|---|---|---|---|
| Cmpd. | ¹H NMR (CDCl₃, 200 MHz) | found | calculated |
| 1 | δ = 7.25~7.33 (3H, m), 7.41 (1H, m), 7.5~7.51 (3H, m), | 371.43 | 371.14 |
| | 7.63 (1H, m), 7.79~7.8 (3H, m), 7.94 (1H, m), | | |
| | 8.05~8.16 (4H, m), 8.55 (1H, m) | | |
| 2 | δ = 7.25~7.33 (3H, m), 7.41 (2H, m), 7.5~7.52 (7H, m), | 447.53 | 447.17 |
| | 7.63 (1H, m), 7.79 (2H, m), 7.94 (1H, m), 8.02 (1H, m), | | |
| | 8.12 (2H, m), 8.45 (1H, m), 8.55 (1H, m) | | |
| 3 | δ = 7.25~7.33 (3H, m), 7.41 (1H, m), 7.51 (2H, m), | 389.42 | 389.13 |
| | 7.61 (1H, m), 7.79~7.8 (3H, m), 7.94 (1H, m), | | |
| | 8.05~8.06 (3H, m), 8.16 (1H, m), 8.55 (1H, m) | | |
| 4 | δ = 7.25 (1H, m), 7.33~7.55 (21H, m), 7.69 (1H, m), | 705.92 | 705.26 |
| | 7.77~7.8 (4H, m), 7.87~7.94 (4H, m), 8.05~8.06 (2H, m), | | |
| | 8.16 (1H, m), 8.55 (1H, m) | | |
| 5 | δ = 7.25 (1H, m), 7.33~7.55 (20H, m), 7.73~7.83 (5H, | 629.82 | 629.23 |
| | m), 7.94 (1H, m), 8.05~8.06 (2H, m), 8.16 (1H, m), | | |
| | 8.55 (1H, m) | | |
| 6 | δ = 1.35 (9H, s), 7.25 (1H, m), 7.33 (1H, m), 7.41 (1H, | 427.54 | 427.20 |
| | m), 7.51~7.55 (3H, m), 7.62 (1H, m), 7.79~7.8 (3H, m), | | |
| | 7.94 (1H, m), 8.05~8.06 (2H, m), 8.16 (1H, m), 8.36 (1H, | | |
| | m), 8.55 (1H, m) | | |
| 7 | δ = 7.41 (3H, m), 7.51~7.52 (10H, m), 7.69 (1H, m), | 523.63 | 523.20 |
| | 7.77~7.8 (5H, m), 7.87 (1H, m), 8~8.06 (3H, m), | | |
| | 8.16~8.18 (2H, m) | | |
| 8 | δ = 1.92 (4H, m), 3.44 (4H, m), 6.88~6.9 (2H, m), | 440.54 | 440.20 |
| | 7.25 (1H, m), 7.33 (1H, m), 7.41~7.51 (4H, m), | | |
| | 7.79~7.8 (3H, m), 7.94 (1H, m), 8.05~8.06 (2H, m), | | |
| | 8.16 (1H, m), 8.55 (1H, m) | | |
| 9 | δ = 7.41 (1H, m), 7.51 (2H, m), 7.57 (2H, m), 7.69 (1H, | 525.60 | 525.20 |
| | m), 7.77~7.8 (5H, m), 7.87 (1H, m), 8~8.06 (3H, m), | | |
| | 8.16~8.18 (2H, m), 8.42 (2H, m), 8.7 (2H, m), 9.24 (2H, | | |
| | m) | | |
| 10 | δ = 5.93 (1H, m), 6.63 (8H, m), 6.75~6.81 (6H, m), | 705.85 | 705.29 |
| | 7.2 (8H, m), 7.38~7.41 (2H, m), 7.51 (2H, m), 7.63 (1H, | | |
| | m), 7.69 (1H, m), 7.79~7.8 (3H, m), 8.05~8.06 (2H, m), | | |
| | 8.16 (1H, m) | | |
| 11 | δ = 7.25~7.33 (3H, m), 7.41 (1H, m), 7.5~7.51 (3H, m), | 447.53 | 447.17 |
| | 7.58~7.68 (4H, m), 7.79~7.84 (6H, m), 7.94 (1H, m), | | |
| | 8.12~8.16 (2H, m), 8.55 (1H, m) | | |
| 12 | δ = 7.25~7.33 (3H, m), 7.41 (1H, m), 7.5~7.51 (3H, m), | 448.52 | 448.17 |
| | 7.58~7.63 (2H, m), 7.79~7.88 (5H, m), 7.94~7.96 (2H, | | |
| | m), 8.12~8.16 (2H, m), 8.55~8.58 (2H, m) | | |
| 13 | δ = 7.25~7.33 (3H, m), 7.41 (1H, m), 7.5~7.51 (3H, m), | 448.52 | 448.17 |
| | 7.58~7.63 (2H, m), 7.72 (1H, m), 7.79~7.86 (5H, m), | | |
| | 7.94 (1H, m), 8.12~8.16 (2H, m), 8.38 (1H, m), 8.55 (1H, | | |
| | m) | | |
| 14 | δ = 7.25~7.33 (3H, m), 7.41 (1H, m), 7.5~7.51 (3H, m), | 449.51 | 449.16 |
| | 7.58~7.63 (2H, m), 7.79~7.84 (4H, m), 7.94 (1H, m), | | |
| | 8.12~8.16 (2H, m), 8.55 (1H, m), 8.79 (1H, s), 8.82 (1H, | | |
| | s) | | |
| 15 | δ = 7.25~7.33 (5H, m), 7.41 (1H, m), 7.5~7.51 (3H, m), | 523.63 | 523.20 |
| | 7.58~7.68 (4H, m), 7.79~7.85 (8H, m), 7.94 (1H, m), | | |
| | 8.12~8.16 (2H, m), 8.55 (1H, m) | | |
| 16 | δ = 7.25~7.33 (3H, m), 7.41 (1H, m), 7.48~7.51 (4H, m), | 523.63 | 523.20 |
| | 7.57~7.7 (6H, m), 7.79~7.84 (6H, m), 7.94 (1H, m), | | |
| | 8.12~8.16 (2H, m), 8.24 (1H, m), 8.55 (1H, m) | | |
| 17 | δ = 1.96 (6H, s), 5.83 (2H, m), 7.25~7.33 (3H, m), | 388.46 | 388.17 |
| | 7.5 (1H, m), 7.63 (1H, m), 7.8 (1H, m), 7.94 (1H, m), | | |
| | 8.05~8.16 (4H, m), 8.55 (1H, m) | | |
| 18 | δ = 2.69 (12H, s), 7.25~7.33 (5H, m), 7.41 (1H, m), | 655.83 | 655.30 |
| | 7.5~7.51 (3H, m), 7.58~7.68 (4H, m), 7.79~7.85 (8H, m), | | |
| | 7.94 (1H, m), 8.12~8.16 (2H, m), 8.55 (1H, m) | | |
| 19 | δ = 2.51 (3H, m), 7.22~7.33 (5H, m), 7.5 (1H, m), | 425.48 | 425.16 |
| | 7.59~7.63 (2H, m), 7.8 (1H, m), 7.94 (1H, m), | | |
| | 8.05~8.16 (4H, m), 8.55~8.56 (2H, m) | | |
| 20 | δ = 1.35 (9H, s), 6.85 (1H, m), 7.13 (1H, s), | 466.58 | 466.22 |
| | 7.25~7.33 (3H, m), 7.43 (1H, m), 7.5 (1H, m), 7.63 (1H, | | |
| | m), 7.72 (1H, m), 7.8 (1H, m), 7.93~7.94 (2H, m), | | |
| | 8.05~8.16 (4H, m), 8.55 (1H, m) | | |
| 21 | δ = 7.25~7.33 (3H, m), 7.5~7.63 (5H, m), 7.7~7.8 (3H, | 504.60 | 504.14 |
| | m), 7.94~8.05 (8H, m), 8.55 (1H, m) | | |
| 22 | δ = 6.95~7.01 (3H, m), 7.25~7.33 (5H, m), 7.5 (1H, m), | 387.43 | 387.14 |
| | 7.63 (1H, m), 7.8 (1H, m), 7.94 (1H, m), 8.05~8.16 (4H, | | |
| | m), 8.55 (1H, m) | | |
| 23 | δ = 7.25~7.33 (3H, m), 7.41~7.51 (12H, m), | 589.69 | 589.23 |
| | 7.58~7.63 (3H, m), 7.79~7.8 (3H, m), 7.94 (1H, m), | | |
| | 8.05~8.16 (4H, m), 8.55 (1H, m) | | |
| 24 | δ = 7.25~7.33 (6H, m), 7.5 (2H, m), 7.63 (2H, m), | 460.53 | 460.17 |
| | 7.8 (1H, m), 7.94 (2H, m), 8.05~8.16 (5H, m), 8.55 (2H, | | |
| | m) | | |
| 25 | δ = 3.83 (3H, m), 7.25~7.33 (7H, m), 7.5 (2H, m), | 490.55 | 490.18 |
| | 7.63 (2H, m), 7.74 (1H, m), 7.94~7.95 (3H, m), 8.12 (2H, | | |
| | m), 8.55 (2H, m) | | |
| 26 | δ = 7.23~7.39 (8H, m), 7.5 (1H, m), 7.63 (1H, m), | 403.50 | 403.11 |
| | 7.8 (1H, m), 7.94 (1H, m), 8.05~8.16 (4H, m), 8.55 (1H, | | |
| | m) | | |
| 27 | δ = 7.25 (1H, m), 7.33 (1H, m), 7.41 (1H, m), | 497.59 | 497.19 |
| | 7.51~7.52 (4H, m), 7.63~7.67 (4H, m), 7.8~7.85 (3H, m), | | |
| | 7.94 (1H, m), 8.05~8.06 (2H, m), 8.16 (2H, m), 8.3 (2H, | | |
| | m), 8.54~8.55 (2H, m) | | |
| 28 | δ = 7.25 (1H, m), 7.33 (1H, m), 7.4~7.41 (2H, m), | 421.49 | 421.16 |
| | 7.51~7.55 (3H, m), 7.67 (2H, m), 7.79~7.8 (3H, m), | | |
| | 7.94 (1H, m), 8.05~8.06 (2H, m), 8.16 (3H, m), 8.55 (1H, | | |
| | m) | | |
| 29 | δ = 7.25 (1H, m), 7.33~7.41 (3H, m), 7.51 (2H, m), | 372.42 | 372.14 |
| | 7.79~7.8 (3H, m), 7.94 (1H, m), 8.05~8.06 (2H, m), | | |
| | 8.16 (1H, m), 8.43 (1H, m), 8.51~8.55 (2H, m) | | |
| 30 | δ = 7.25~7.37 (20H, m), 7.61~7.63 (2H, m), | 629.82 | 629.23 |
| | 7.76~7.8 (2H, m), 7.89~7.94 (2H, m), 8.05~8.16 (4H, m), | | |
| | 8.55 (1H, m) | | |
| 31 | δ = 6.51 (2H, m), 6.69 (2H, m), 6.98~7.01 (4H, m), | 537.65 | 537.22 |
| | 7.11 (4H, m), 7.26 (2H, m), 7.33 (4H, m), 7.41 (1H, m), | | |
| | 7.51 (2H, m), 7.58 (1H, m), 7.79~7.84 (4H, m), 8.16 (1H, | | |
| | m) | | |
| 32 | δ = 6.73 (2H, m), 6.91 (2H, m), 7.39~7.41 (3H, m), | 399.44 | 399.14 |
| | 7.51~7.58 (5H, m), 7.79~7.84 (4H, m), 8.16 (1H, m) | | |
| 33 | δ = 6.59 (2H, m), 6.77 (2H, m), 6.89~6.92 (4H, m), | 387.43 | 387.14 |
| | 7.41 (1H, m), 7.51 (2H, m), 7.58 (1H, m), 7.79~7.84 (4H, | | |
| | m), 8.16 (1H, m) | | |
| 34 | δ = 6.97 (2H, m), 7.16~7.21 (6H, m), 7.41 (1H, m), | 403.50 | 403.11 |
| | 7.51 (2H, m), 7.58 (1H, m), 7.79~7.84 (4H, m), 8.16 (1H, | | |
| | m) | | |
| 35 | δ = 6.38 (4H, m), 6.56 (4H, m), 6.63 (2H, m), 6.81 (1H, | 462.54 | 462.18 |
| | m), 7.2 (2H, m), 7.41 (1H, m), 7.51 (2H, m), 7.58 (1H, | | |
| | m), 7.79~7.84 (4H, m), 8.16 (1H, m) | | |
| 36 | δ = 6.63 (2H, m), 6.81 (2H, m), 6.99~7.05 (4H, m), | 397.47 | 397.16 |
| | 7.25 (2H, m), 7.41 (1H, m), 7.51 (2H, m), 7.58 (1H, m), | | |
| | 7.79~7.84 (4H, m), 8.16 (1H, m) | | |
| 37 | δ = 7.25~7.33 (3H, m), 7.41 (1H, m), 7.5~7.52 (5H, m), | 447.53 | 447.17 |
| | 7.63 (1H, m), 7.8~7.85 (3H, m), 7.94 (1H, m), | | |
| | 8.05~8.16 (4H, m), 8.3 (2H, m), 8.55 (1H, m) | | |
| 38 | δ = 7.14 (1H, m), 7.25~7.33 (3H, m), 7.5 (1H, m), | 449.51 | 449.16 |
| | 7.63 (1H, m), 7.7 (1H, m), 7.8 (1H, m), 7.88 (1H, m), | | |
| | 7.94 (1H, m), 8.05~8.16 (4H, m), 8.53~8.55 (2H, m), | | |
| | 8.81 (1H, m), 8.99 (1H, m), 9.3 (1H, m) | | |
| 39 | δ = 7.25~7.33 (3H, m), 7.5~7.55 (3H, m), 7.61~7.63 (2H, | 421.49 | 421.16 |
| | m), 7.8 (1H, m), 7.94~7.95 (2H, m), 8.04~8.16 (6H, m), | | |
| | 8.55 (2H, m) | | |
| 40 | δ = 7.25~7.33 (3H, m), 7.42 (1H, m), 7.49~7.5 (2H, m), | 422.48 | 422.15 |
| | 7.63 (1H, m), 7.76~7.8 (2H, m), 7.92~7.94 (2H, m), | | |
| | 8.05~8.16 (4H, m), 8.43 (1H, m), 8.55 (1H, m), 8.87 (1H, | | |
| | m) | | |
| 41 | δ = 7.25~7.33 (3H, m), 7.5 (1H, m), 7.63 (1H, m), | 471.55 | 471.17 |
| | 7.8~7.94 (7H, m), 8.05~8.16 (6H, m), 8.55 (1H, m), | | |
| | 8.93 (2H, m) | | |
| 42 | δ = 7.25~7.33 (3H, m), 7.5 (1H, m), 7.58~7.65 (4H, m), | 473.53 | 473.16 |
| | 7.8 (1H, m), 7.94 (1H, m), 8.05~8.16 (4H, m), 8.38 (2H, | | |
| | m), 8.55 (1H, m), 8.83 (2H, m) | | |
| 43 | δ = 7.25~7.33 (3H, m), 7.5 (1H, m), 7.63 (1H, m), | 521.61 | 521.19 |
| | 7.8~7.88 (5H, m), 7.94 (1H, m), 8.04~8.18 (8H, m), | | |
| | 8.55 (1H, m), 8.93 (2H, m), 9.15 (1H, m) | | |
| 44 | δ = 2.34 (3H, m), 7.25~7.33 (5H, m), 7.5 (1H, m), | 385.46 | 385.16 |
| | 7.63~7.67 (3H, m), 7.8 (1H, m), 7.94 (1H, m), | | |
| | 8.05~8.16 (4H, m), 8.55 (1H, m) | | |
| 45 | δ = 7.25~7.36 (4H, m), 7.5 (1H, m), 7.63 (1H, m), | 372.42 | 372.14 |
| | 7.8~7.85 (2H, m), 7.94 (1H, m), 8.05~8.16 (4H, m), | | |
| | 8.4 (1H, m), 8.55~8.59 (2H, m) | | |
| 46 | δ = 7.25~7.33 (3H, m), 7.41 (2H, m), 7.5~7.51 (5H, m), | 601.70 | 601.23 |
| | 7.57~7.63 (2H, m), 7.7~7.8 (7H, m), 7.94 (1H, m), | | |
| | 8.05~8.16 (4H, m), 8.23 (1H, s), 8.24 (1H, m), 8.55 (1H, | | |
| | m) | | |
| 47 | δ = 2.44 (6H, s), 7.25~7.33 (3H, m), 7.5 (1H, m), | 402.45 | 402.16 |
| | 7.63 (1H, m), 7.8 (1H, m), 7.94 (1H, m), 8.05~8.16 (4H, | | |
| | m), 8.55 (1H, m) | | |
| 48 | δ = 7.25~7.33 (3H, m), 7.41 (2H, m), 7.5~7.51 (5H, m), | 526.59 | 526.19 |
| | 7.63 (1H, m), 7.8 (1H, m), 7.94 (1H, m), 8.05~8.16 (4H, | | |
| | m), 8.28 (4H, m), 8.55 (1H, m) | | |
| 49 | δ = 7.25~7.33 (3H, m), 7.42~7.43 (3H, m), 7.5~7.55 (3H, | 395.45 | 395.14 |
| | m), 7.63 (1H, m), 7.8 (1H, m), 7.94 (1H, m), | | |
| | 8.05~8.16 (4H, m), 8.55 (1H, m) | | |
| 50 | δ = 6.99 (2H, m), 7.25~7.33 (3H, m), 7.41 (2H, m), | 552.63 | 552.21 |
| | 7.5~7.51 (5H, m), 7.63 (1H, m), 7.8 (1H, m), 7.94 (1H, | | |
| | m), 8.05~8.16 (4H, m), 8.28 (4H, m), 8.55 (1H, m) | | |
| 51 | δ = 3.57 (4H, m), 3.65 (4H, m), 7.25~7.33 (3H, m), | 380.44 | 380.16 |
| | 7.5 (1H, m), 7.63 (1H, m), 7.8 (1H, m), 7.94 (1H, m), | | |
| | 8.05~8.16 (4H, m), 8.55 (1H, m) | | |
| 52 | δ = 7.25~7.37 (9H, m), 7.46~7.55 (12H, m), 7.63 (1H, | 705.92 | 705.26 |
| | m), 7.8~7.94 (6H, m), 8.05~8.16 (4H, m), 8.3 (2H, m), | | |
| | 8.55 (1H, m) | | |
| 53 | δ = 7.25~7.37 (10H, m), 7.46~7.55 (10H, m), 7.63 (1H, | 711.95 | 711.22 |
| | m), 7.73 (1H, m), 7.8~7.85 (3H, m), 7.94 (1H, m), | | |
| | 8.05~8.16 (4H, m), 8.3 (2H, m), 8.55 (1H, m) | | |
| 54 | δ = 6.63 (4H, m), 6.81 (2H, m), 7.2~7.33 (7H, m), | 462.54 | 462.18 |
| | 7.5 (1H, m), 7.63 (1H, m), 7.8 (1H, m), 7.94 (1H, m), | | |
| | 8.05~8.16 (4H, m), 8.55 (1H, m) | | |
| 55 | δ = 7.25~7.33 (6H, m), 7.41 (2H, m), 7.5~7.51 (6H, m), | 612.72 | 612.23 |
| | 7.63 (2H, m), 7.79 (4H, m), 7.94 (2H, m), 8.12 (2H, m), | | |
| | 8.34 (1H, s), 8.51 (1H, s), 8.55 (2H, m) | | |
| 56 | δ = 3.83 (2H, s), 7.25~7.33 (6H, m), 7.46 (1H, s), | 520.58 | 520.19 |
| | 7.5 (2H, m), 7.63 (3H, m), 7.63 (0H, s), 7.94 (2H, m), | | |
| | 8.12 (2H, m), 8.55 (2H, m) | | |
| 57 | δ = 6.95~7.01 (3H, m), 7.25~7.33 (5H, m), 7.5 (1H, m), | 463.53 | 463.17 |
| | 7.58~7.68 (4H, m), 7.79~7.84 (4H, m), 7.94 (1H, m), | | |
| | 8.12~8.16 (2H, m), 8.55 (1H, m) | | |
| 58 | δ = 4.28 (4H, m), 7.2~7.33 (7H, m), 7.5~7.51 (3H, m), | 518.56 | 518.17 |
| | 7.63 (2H, m), 7.94 (2H, m), 8.12 (2H, m), 8.55 (2H, m) | | |
| 59 | δ = 3.83 (3H, m), 7.25~7.33 (7H, m), 7.5 (2H, m), | 490.55 | 490.18 |
| | 7.63 (2H, m), 7.74 (1H, m), 7.94~7.95 (3H, m), 8.12 (2H, | | |
| | m), 8.55 (2H, m) | | |
| 60 | δ = 7.14~7.17 (3H, m), 7.25~7.33 (6H, m), 7.41~7.5 (5H, | 552.62 | 552.20 |
| | m), 7.63 (2H, m), 7.89~7.94 (3H, m), 8.02 (1H, m), | | |
| | 8.12 (2H, m), 8.55 (2H, m) | | |
| 61 | δ = 2.44 (3H, m), 7.25~7.33 (3H, m), 7.5 (1H, m), | 309.36 | 309.13 |
| | 7.63 (1H, m), 7.8 (1H, m), 7.94 (1H, m), 8.05~8.16 (4H, | | |
| | m), 8.55 (1H, m) | | |
| 62 | δ = 3.83 (3H, s), 5.35 (1H, s), 7.25~7.33 (3H, m), | 341.36 | 341.12 |
| | 7.42 (1H, s), 7.5 (1H, m), 7.57 (1H, s), 7.63 (1H, m), | | |
| | 7.94 (1H, m), 8.12 (1H, m), 8.55 (1H, m), 9.36 (1H, s) | | |
| 63 | δ = 5.35 (1H, s), 7.25~7.33 (4H, m), 7.5 (1H, m), | 311.34 | 311.11 |
| | 7.63 (1H, m), 7.7 (1H, m), 7.89~7.94 (2H, m), 8.12 (1H, | | |
| | m), 8.55 (1H, m), 9.36 (1H, s) | | |
| 64 | δ = 7.25~7.33 (6H, m), 7.5 (2H, m), 7.63 (2H, m), | 505.53 | 505.15 |
| | 7.94 (2H, m), 8.12 (2H, m), 8.32 (1H, m), 8.55 (2H, m), | | |
| | 8.73 (1H, m), 9.16 (1H, m) | | |
| 65 | δ = 7.25~7.33 (3H, m), 7.41~7.51 (6H, m), | 447.53 | 447.17 |
| | 7.58~7.63 (2H, m), 7.79~7.84 (4H, m), 7.94 (1H, m), | | |
| | 8.09~8.16 (3H, m), 8.28 (1H, m), 8.55 (1H, m) | | |
| 66 | δ = 1.72 (6H, s), 7.25~7.38 (5H, m), 7.5~7.55 (2H, m), | 487.59 | 487.20 |
| | 7.63 (2H, m), 7.77~7.8 (2H, m), 7.87~7.94 (3H, m), | | |
| | 8.05~8.16 (4H, m), 8.55 (1H, m) | | |
| 67 | δ = 7.37~7.58 (23H, m), 7.72 (2H, m), 7.79~7.84 (9H, | 744.95 | 744.27 |
| | m), 8.16 (2H, m) | | |
| 68 | δ = 7.41 (3H, m), 7.51 (6H, m), 7.58 (3H, m), 7.66 (3H, | 690.79 | 690.25 |
| | m), 7.79~7.84 (12H, m), 8.16 (3H, m) | | |
| 69 | δ = 7.25~7.41 (5H, m), 7.5~7.51 (3H, m), 7.63 (1H, m), | 372.42 | 372.14 |
| | 7.79 (2H, m), 7.94 (1H, m), 8.12 (1H, m), 8.43 (1H, m), | | |
| | 8.51~8.55 (2H, m) | | |
| 72 | δ = 7.25~7.36 (7H, m), 7.5 (2H, m), 7.63~7.68 (6H, m), | 613.71 | 613.23 |
| | 7.79 (4H, m), 7.94 (2H, m), 8.12 (2H, m), 8.43 (1H, m), | | |
| | 8.51~8.55 (3H, m) | | |
| 75 | δ = 7.25~7.38 (7H, m), 7.5 (2H, m), 7.63 (2H, m), | 461.52 | 461.16 |
| | 7.94 (2H, m), 8.12 (2H, m), 8.43 (1H, m), 8.55 (2H, m), | | |
| | 8.87 (1H, m) | | |
| 76 | δ = 7.25~7.41 (5H, m), 7.5~7.51 (3H, m), 7.63~7.68 (3H, | 448.52 | 448.17 |
| | m), 7.79 (4H, m), 7.94 (1H, m), 8.12 (1H, m), 8.43 (1H, | | |
| | m), 8.55 (1H, m), 8.87 (1H, m) | | |
| 79 | δ = 1.35 (9H, s), 7.25~7.33 (3H, m), 7.41 (1H, m), | 428.53 | 428.20 |
| | 7.5~7.52 (5H, m), 7.63 (1H, m), 7.94 (1H, m), 8.12 (1H, | | |
| | m), 8.55 (1H, m), 8.98 (1H, m), 9.24 (1H, m) | | |
| 85 | δ = 2.34 (12H, s), 7.25~7.33 (5H, m), 7.5 (1H, m), | 528.65 | 528.23 |
| | 7.6~7.67 (5H, m), 7.94 (1H, m), 8.12 (1H, m), 8.55 (1H, | | |
| | m), 8.79 (1H, m), 8.95 (1H, m) | | |
| 89 | δ = 7.25~7.33 (3H, m), 7.5~7.51 (2H, m), 7.63~7.68 (3H, | 448.52 | 448.17 |
| | m), 7.79~7.85 (4H, m), 7.94 (1H, m), 8.12 (1H, m), | | |
| | 8.3 (2H, m), 8.43 (1H, m), 8.55 (1H, m), 9.27 (1H, s), | | |
| | 9.51 (1H, m) | | |
| 93 | δ = 3.83 (6H, s), 6.97 (1H, m), 7.25~7.33 (3H, m), | 508.57 | 508.19 |
| | 7.41~7.51 (6H, m), 7.62~7.63 (2H, m), 7.76 (1H, m), | | |
| | 7.9~7.97 (3H, m), 8.12 (1H, m), 8.55 (1H, m) | | |
| 98 | δ = 7.22~7.33 (4H, m), 7.5 (1H, m), 7.63 (1H, m), | 296.33 | 296.11 |
| | 7.94~7.97 (2H, m), 8.12 (1H, m), 8.43 (1H, m), 8.55 (1H, | | |
| | m), 8.87 (1H, s) | | |
| 104 | δ = 7.25 (4H, m), 7.29 (5H, s), 7.29~7.33 (4H, m), | 791.90 | 791.28 |
| | 7.5 (4H, m), 7.63 (4H, m), 7.94 (4H, m), 8.12 (4H, m), | | |
| | 8.55 (4H, m) | | |
| 108 | δ = 7.25~7.33 (3H, m), 7.46~7.51 (3H, m), 7.63 (1H, m), | 373.41 | 373.13 |
| | 7.79 (1H, m), 7.94 (1H, m), 8.09~8.12 (2H, m), 8.55 (1H, | | |
| | m), 8.78 (1H, m), 9.26 (1H, m), 9.27 (1H, s) | | |
| 112 | δ = 7.25~7.33 (3H, m), 7.41~7.51 (6H, m), 7.63 (1H, m), | 441.41 | 441.12 |
| | 7.94 (1H, m), 8.12 (1H, m), 8.55 (1H, m), 8.57 (1H, s) | | |
| 115 | δ = 7.25~7.33 (3H, m), 7.5 (1H, m), 7.63~7.68 (3H, m), | 373.41 | 373.13 |
| | 7.79 (2H, m), 7.94 (1H, m), 8.12 (1H, m), 8.5 (1H, s), | | |
| | 8.55 (1H, m), 8.63 (2H, m) | | |
| 121 | δ = 1.53 (4H, m), 1.59 (2H, m), 4.03 (4H, m), | 621.73 | 621.26 |
| | 7.25~7.33 (6H, m), 7.41 (1H, m), 7.5~7.51 (4H, m), | | |
| | 7.63 (2H, m), 7.79 (2H, m), 7.94 (2H, m), 8.12 (2H, m), | | |
| | 8.55 (2H, m) | | |
| 124 | δ = 7.25 (1H, m), 7.33~7.37 (8H, m), 7.46 (6H, m), | 555.70 | 555.19 |
| | 7.55 (3H, m), 7.73 (1H, m), 7.83 (1H, m), 7.94 (1H, m), | | |
| | 8.55 (1H, m), 8.63 (2H, m), 8.87 (1H, s) | | |
| 125 | δ = 7.25~7.37 (20H, m), 7.61~7.63 (2H, m), 7.76 (1H, | 631.80 | 631.22 |
| | m), 7.89~7.94 (2H, m), 8.12 (1H, m), 8.55 (1H, m), | | |
| | 8.63 (2H, m) | | |
| 126 | δ = 7.25~7.33 (3H, m), 7.41~7.51 (6H, m), | 449.51 | 449.16 |
| | 7.63~7.68 (3H, m), 7.79 (2H, m), 7.94 (1H, m), 8.12 (1H, | | |
| | m), 8.55 (1H, m), 8.63 (2H, m) | | |
| 130 | δ = 7.25 (2H, m), 7.33 (2H, m), 7.41~7.51 (10H, m), | 714.81 | 714.25 |
| | 7.63~7.67 (8H, m), 7.94 (2H, m), 8.16 (2H, m), | | |
| | 8.54~8.55 (4H, m) | | |
| 132 | δ = 7.25 (2H, m), 7.33 (2H, m), 7.41~7.51 (12H, m), | 616.67 | 616.21 |
| | 7.94 (2H, m), 8.43 (2H, m), 8.55 (2H, m), 9.34 (2H, m) | | |
| 139 | δ = 7.25~7.33 (3H, m), 7.41~7.51 (17H, m), | 766.89 | 766.28 |
| | 7.58~7.63 (5H, m), 7.69 (1H, m), 7.77 (2H, m), 7.87 (1H, | | |
| | m), 7.94~8 (2H, m), 8.12 (1H, m), 8.18 (1H, m), | | |
| | 8.55 (1H, m) | | |
| 141 | δ = 7.37 (3H, m), 7.57 (3H, m), 7.69~7.75 (6H, m), | 398.46 | 398.15 |
| | 7.94 (3H, m), 8.22 (3H, m) | | |
| 142 | δ = 7.25~7.33 (7H, m), 7.41 (1H, m), 7.5~7.52 (5H, m), | 446.54 | 446.18 |
| | 7.6~7.63 (2H, m), 7.68 (1H, s), 7.78 (1H, m), | | |
| | 7.94~7.98 (2H, m), 8.06~8.12 (2H, m), 8.55 (1H, m) | | |
| 144 | δ = 1.35 (3H, s), 7.25~7.38 (7H, m), 7.5 (1H, m), | 426.55 | 426.21 |
| | 7.6~7.63 (2H, m), 7.78 (1H, m), 7.94~7.98 (2H, m), | | |
| | 8.06~8.12 (2H, m), 8.42 (1H, m), 8.55 (1H, m) | | |
| 146 | δ = 7.25~7.33 (6H, m), 7.42 (2H, m), 7.49~7.5 (3H, m), | 459.54 | 459.17 |
| | 7.63 (3H, m), 7.92~7.94 (3H, m), 8.12 (2H, m), 8.55 (2H, | | |
| | m) | | |
| 147 | δ = 7.25~7.33 (3H, m), 7.42 (1H, m), 7.49~7.5 (2H, m), | 472.54 | 472.17 |
| | 7.58~7.65 (4H, m), 7.76 (1H, m), 7.92~7.94 (2H, m), | | |
| | 8.12 (1H, m), 8.38 (2H, m), 8.43 (1H, s), 8.55 (1H, m), | | |
| | 8.83 (2H, m) | | |
| 148 | δ = 7.25~7.37 (20H, m), 7.61~7.63 (2H, m), | 629.82 | 629.23 |
| | 7.76~7.8 (2H, m), 7.89~7.94 (2H, m), 8.05~8.16 (4H, m), | | |
| | 8.55 (1H, m) | | |
| 149 | δ = 7.25 (1H, m), 7.33~7.46 (14H, m), 7.55 (3H, m), | 679.88 | 679.24 |
| | 7.61~7.67 (5H, m), 7.76~7.8 (2H, m), 7.89~7.94 (2H, m), | | |
| | 8.05~8.06 (2H, m), 8.16 (2H, m), 8.54~8.55 (2H, m) | | |
| 150 | δ = 7.25 (1H, m), 7.28~7.41 (22H, m), 7.68 (1H, m), | 705.92 | 705.26 |
| | 7.8~7.85 (3H, m), 7.94 (1H, m), 8.05~8.06 (2H, m), | | |
| | 8.16 (1H, m), 8.22 (1H, m), 8.3 (2H, m), 8.55 (1H, m) | | |
| 151 | δ = 7.25 (1H, m), 7.33 (1H, m), 7.41 (1H, m), | 573.68 | 573.22 |
| | 7.51~7.52 (4H, m), 7.58~7.68 (7H, m), 7.79~7.85 (6H, | | |
| | m), 7.94 (1H, m), 8.16 (2H, m), 8.3 (2H, m), | | |
| | 8.54~8.55 (2H, m) | | |
| 152 | δ = 7.25 (1H, m), 7.29 (1H, m), 7.33~7.46 (22H, m), | 782.01 | 781.29 |
| | 7.63~7.68 (3H, m), 7.79~7.85 (4H, m), 7.94 (1H, m), | | |
| | 8.11~8.12 (3H, m), 8.26~8.3 (3H, m), 8.55 (1H, m) | | |
| 153 | δ = 7.25 (1H, m), 7.28 (1H, m), 7.33~7.46 (22H, m), | 782.01 | 781.29 |
| | 7.68 (3H, m), 7.79~7.85 (6H, m), 7.94 (1H, m), 8.16 (1H, | | |
| | m), 8.22 (1H, m), 8.3 (2H, m), 8.55 (1H, m) | | |
| 154 | δ = 0.66 (3H, m), 7.25 (1H, m), 7.28~7.41 (17H, m), | 643.85 | 643.24 |
| | 7.68 (1H, m), 7.8~7.85 (3H, m), 7.94 (1H, m), | | |
| | 8.05~8.06 (2H, m), 8.16 (1H, m), 8.22 (1H, m), 8.3 (2H, | | |
| | m), 8.55 (1H, m) | | |
| 155 | δ = 7.25 (5H, m), 7.33 (1H, m), 7.41 (1H, m), | 496.60 | 496.19 |
| | 7.51~7.52 (4H, m), 7.6~7.67 (5H, m), 7.68 (1H, s), | | |
| | 7.78 (1H, m), 7.94~7.98 (2H, m), 8.06 (1H, m), 8.16 (1H, | | |
| | m), 8.54~8.55 (2H, m) | | |
| 156 | δ = 7.25 (4H, m), 7.36 (1H, m), 7.37 (6H, m), 7.41 (1H, | 754.99 | 754.28 |
| | m), 7.46~7.55 (18H, m), 7.68 (1H, s), 7.78~7.83 (2H, | | |
| | m), 7.98~8.06 (3H, m), 8.16 (1H, m), 8.54 (1H, m) | | |
| 157 | δ = 0.66 (3H, m), 7.25~7.28 (5H, m), 7.37 (4H, m), | 692.92 | 692.26 |
| | 7.41 (1H, m), 7.46 (4H, m), 7.51~7.6 (13H, m), 7.68 (0H, | | |
| | s), 7.78 (1H, m), 7.98 (1H, m), 8.06 (1H, m), 8.16 (1H, | | |
| | m), 8.53~8.54 (2H, m) | | |
| 158 | δ = 0.66 (3H, m), 7.28 (1H, m), 7.37~7.55 (15H, m), | 693.91 | 693.26 |
| | 7.63~7.68 (5H, m), 7.8~7.85 (3H, m), 8.05~8.06 (2H, m), | | |
| | 8.16 (2H, m), 8.3 (2H, m), 8.53~8.54 (2H, m) | | |
| 159 | δ = 0.66 (3H, m), 7.36~7.55 (16H, m), 7.63~7.67 (4H, | 693.91 | 693.26 |
| | m), 7.8~7.85 (4H, m), 8.04~8.06 (3H, m), 8.16 (2H, m), | | |
| | 8.3 (2H, m), 8.54 (1H, m) | | |

### Example 1-8

Manufacture of OLED Device Using the Organic Electroluminescent Compound According to the Present Invention

An OLED device was manufactured using the electroluminescent material according to the present invention. First, a transparent electrode ITO thin film (15 Ω/□) obtained from a glass for OLED (produced by Samsung Corning) was subjected to ultrasonic washing with trichloroethylene, acetone, ethanol and distilled water, sequentially, and stored in isopropanol before use. Then, an ITO substrate was equipped in a substrate folder of a vacuum vapor deposition apparatus, and 4,4′,4″-tris(N,N-(2-naphthyl)-phenylamino)triphenylamine (2-TNATA) was placed in a cell of the vacuum vapor deposition apparatus, which was then ventilated up to 10⁻⁶ torr of vacuum in the chamber. Then, electric current was applied to the cell to evaporate 2-TNATA, thereby forming a hole injection layer having a thickness of 60 nm on the ITO substrate.

Then, N,N′-bis(α-naphthyl)-N,N′-diphenyl-4,4′-diamine (NPB) was placed in another cell of the vacuum vapor deposition apparatus, and electric current was applied to the cell to evaporate NPB, thereby forming a hole transport layer having a thickness of 20 nm on the hole injection layer.

An electroluminescent layer was formed on the hole transport layer as follows. The compound according to the present invention (e.g., Compound 52) vacuum sublimed at 10⁻⁶ torr was filled in a cell of a vacuum vapor deposition apparatus as a host material, and an electroluminescent dopant (e.g., Compound of (piq)₂Ir(acac)[bis-(1-phenylisoquinolyl)iridium(III)acetylacetonate]) was filled in another cell. The two materials were evaporated at different speed, so that an electroluminescent layer having a thickness of 30 nm was formed on the hole transport layer at 4 to 10 mol %.

Then, tris(8-hydroxyquinoline)-aluminum(III) (Alq) was vapor-deposited as an electron transport layer with a thickness of 20 nm, and lithium quinolate (Liq) was vapor-deposited as an electron injecting layer with a thickness of 1 to 2 nm. Thereafter, an Al cathode was vapor-deposited with a thickness of 150 nm by using another vacuum vapor-deposit device to manufacture an OLED.

Table 2 shows the result of driving voltage (V) and luminous efficiency (cd/A) according to Examples.

### Comparative Example 1

Manufacture of an OLED Using Conventional Electroluminescent Material

An OLED was manufactured in the same manner as Example 1 except that 4,4′-di(9H-carbazol-9-one)biphenyl (CBP) instead of the organic electroluminescent compound according to the present invention was used as electroluminescent host material in another cell of the vacuum vapor deposition device and Bis(2-methyl-8-quinolinato)(p-phenylphenolato)-aluminum(III)(BAlq) was used as an electron transport layer.

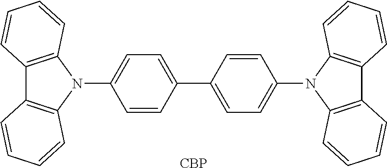

The driving voltage and the luminous efficiencies of the OLED comprising the organic electroluminescent compound according to the present invention (Examples 1 to 5) or conventional EL compounds (Comparative Example 1) were measured at 1,000 cd/m², respectively, and the results are shown in Table 2.

TABLE 2

| | | | @1,000 cd/m² | | |
|---|---|---|---|---|---|
| | | | Driving | Luminous | |
| | Host | Luminous | voltage | efficiency | |
| | material | material | (V) | (cd/A) | color |
| Example 1 | Compound | (piq)₂Ir(acac) | 6.8 | 7.2 | red |
| | 27 | | | | |
| Example 2 | Compound | (piq)₂Ir(acac) | 6.7 | 7.4 | red |
| | 37 | | | | |
| Example 3 | Compound | (piq)₂Ir(acac) | 7.0 | 7.7 | red |
| | 52 | | | | |
| Example 4 | Compound | (piq)₂Ir(acac) | 6.6 | 7.1 | red |
| | 77 | | | | |
| Example 5 | Compound | (piq)₂Ir(acac) | 6.6 | 7.2 | red |
| | 143 | | | | |
| Example 6 | Compound | (piq)₂Ir(acac) | 6.8 | 7.6 | red |
| | 153 | | | | |
| Example 7 | Compound | (piq)₂Ir(acac) | 6.9 | 7.5 | red |
| | 154 | | | | |
| Example 8 | Compound | (piq)₂Ir(acac) | 6.6 | 7.4 | red |
| | 155 | | | | |
| Example 9 | Compound | (piq)₂Ir(acac) | 6.9 | 7.8 | red |
| | 156 | | | | |
| Comparative | CBP | (piq)₂Ir(acac) | 7.5 | 6.5 | red |
| Example 1 | | | | | |

As shown in Table 2, the organic electroluminescent compounds according to the present invention have excellent luminous properties compared with the conventional material. In addition, the device using the organic electroluminescent compound according to the present invention as host material for emitting red color has excellent luminescent efficiency and drops driving voltage, thereby reducing power consumption.

what is claimed is:

1. An organic electroluminescent compound represented by Chemical Formula 1: 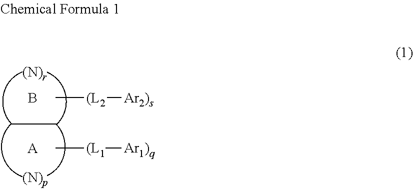
wherein
ring A and ring B represent a 6-membered aromatic ring or a 6-membered hetero aromatic ring;
p and r represent an integer from 0 to 2, and q and s represent an integer from 2 to 4, with the proviso that p+r≧1, p+q=4 and r+s=4;
L₁ and L₂ independently represent a chemical bond, (C6-C30)arylene with or without substituent(s), (C3-C30)heteroarylene with or without substituent(s), 5- to 7-membered heterocycloalkylene with or without substituent(s), 5- to 7-membered heterocycloalkylene fused with one or more aromatic ring(s) with or without substituent(s), (C3-C30)cycloalkylene with or without substituent(s), (C3-C30)cycloalkylene fused with one or more aromatic ring(s) with or without substituent(s), (C2-C30)alkenylene with or without substituent(s), (C2-C30)alkynylene with or without substituent(s), (C6-C30)ar(C1-C30)alkylene with or without substituent(s), —O— or —S—;
Ar₁ and Ar₂ independently represent hydrogen, halogen, (C1-C30)alkyl with or without substituent(s), (C6-C30)aryl with or without substituent(s), substituted or unsubstituted (C6-C30)aryl fused with one or more (C3-C30)cycloalkyl with or without substituent(s), (C3-C30)heteroaryl with or without substituent(s), 5- to 7-membered heterocycloalkyl with or without substituent(s), 5- to 7-membered heterocycloalkyl fused with one or more aromatic ring(s) with or without substituent(s), (C3-C30)cycloalkyl with or without substituent(s), (C3-C30)cycloalkyl fused with one or more aromatic ring(s) with or without substituent(s), cyano, —NR₁₁R₁₂, —SiR₁₃R₁₄R₁₅, —OR₁₆, —SR₁₇, (C6-C30)ar(C1-C30)alkyl with or without substituent(s), (C1-C30)alkylamino with or without substituent(s), (C3-C30)heteroaryl with or without —SiR₁₈R₁₉R₂₀ substituent(s), (C6-C30)arylamino with or without substituent(s), (C2-C30)alkenyl with or without substituent(s), ((C2-C30)alkynyl with or without substituent(s), carboxyl, nitro or hydroxyl, or each of them may be linked to an adjacent substituent via (C3-C30)alkylene or (C3-C30)alkenylene with or without a fused ring to form an alicylic ring or a mono- or polycyclic aromatic ring;
the R₁₁ through R₂₀ independently represent (C1-C30)alkyl with or without substituent(s), (C6-C30)aryl with or without substituent(s) or (C3-C30)heteroaryl with or without substituent(s) or each of them may be linked to an adjacent substituent via (C3-C30)alkylene or (C3-C30)alkenylene with or without a fused ring to form an alicylic ring or a mono- or polycyclic aromatic ring;
the heterocycloalkyl and heteroaryl may include one or more heteroatom(s) selected from N, O, S and Si.

2. The organic electroluminescent compound according to claim 1, wherein in “with or without substituents” of the R₁₁ through R₂₀, L₁, L₂, Ar₁ and Ar₂, the substituent is further substituted by one or more substituent(s) independently selected from deuterium, halogen, (C1-C30)alkyl with or without halogen substituent(s), (C6-C30)aryl, (C3-C30)heteroaryl with or without (C6-C30)aryl substituent(s), 5- to 7-membered heterocycloalkyl, 5- to 7-membered heterocycloalkyl fused with one or more aromatic rings, (C3-C30)cycloalkyl, (C3-C30)cycloalkyl fused with one or more aromatic rings, tri(C1-C30)alkylsilyl, di(C1-C30)alkyl(C6-C30)arylsilyl, tri(C6-C30)arylsilyl, (C2-C30)alkenyl, (C2-C30)alkynyl, cyano, carbazolyl, —NR₃₁R₃₂, —SiR₃₃R₃₄R₃₅, —OR₃₆, —SR₃₇, (C6-C30)ar(C1-C30)alkyl, (C1-C30)alkyl(C6-C30)aryl, (C1-C30)alkyloxy, (C1-C30)alkylthio, (C6-C30)aryloxy, (C6-C30)arylthio, carboxyl, nitro or hydroxyl, or is linked to an adjacent substituent to form a ring,
the R₃₁ through R₃₇ are linked to (C1-C30)alkyl, (C6-C30)aryl, (C3-C30)heteroaryl or an adjacent substituent via (C3-C30)alkylene or (C3-C30)alkenylene with or without a fused ring to form an alicylic ring or a mono- or polycyclic aromatic ring.

3. The organic electroluminescent compound according to claim 1, which is represented by Chemical Formulas 2 to 5: 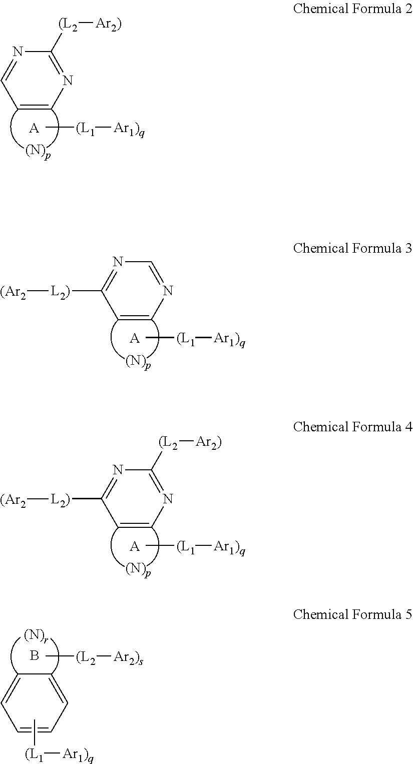
wherein
a ring A, a ring B, Ar₁, Ar₂, p, q, r and s are the same as defined in claim 1, wherein each of the substituents may be different from each other.

4. The organic electroluminescent compound according to claim 1, which is selected from the following compounds: 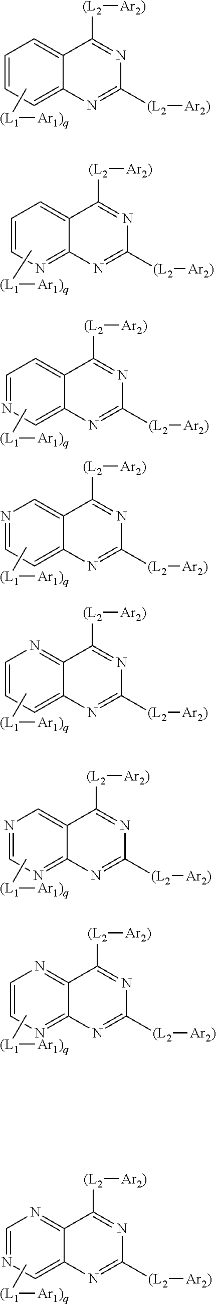 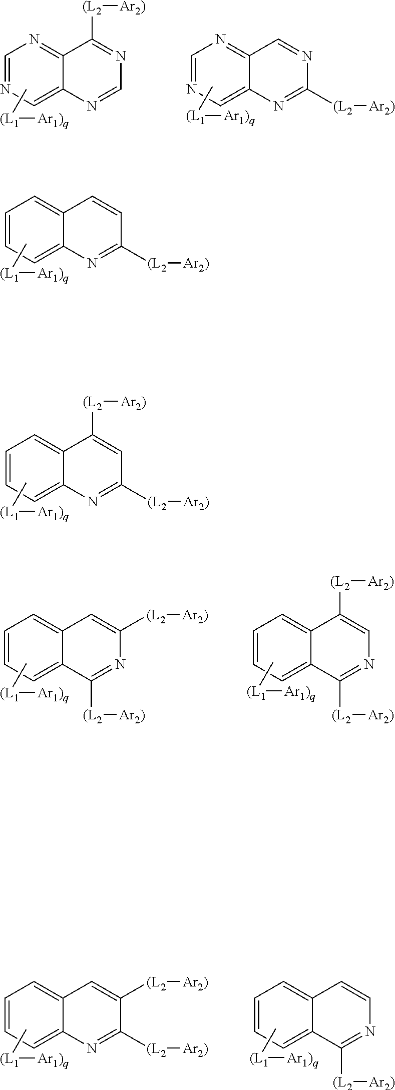
wherein
L₁, L₂, Ar₁, Ar₂ and q are the same as defined in claim 1, wherein each of the substituents may be different from each other.

5. The organic electroluminescent compound according to claim 4, wherein Ar₁and Ar₂ are selected from the following structures:

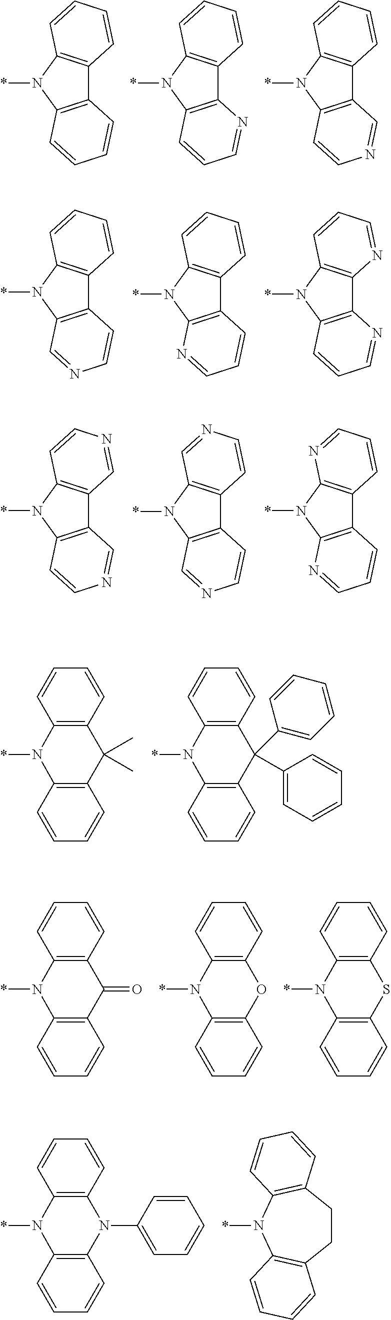

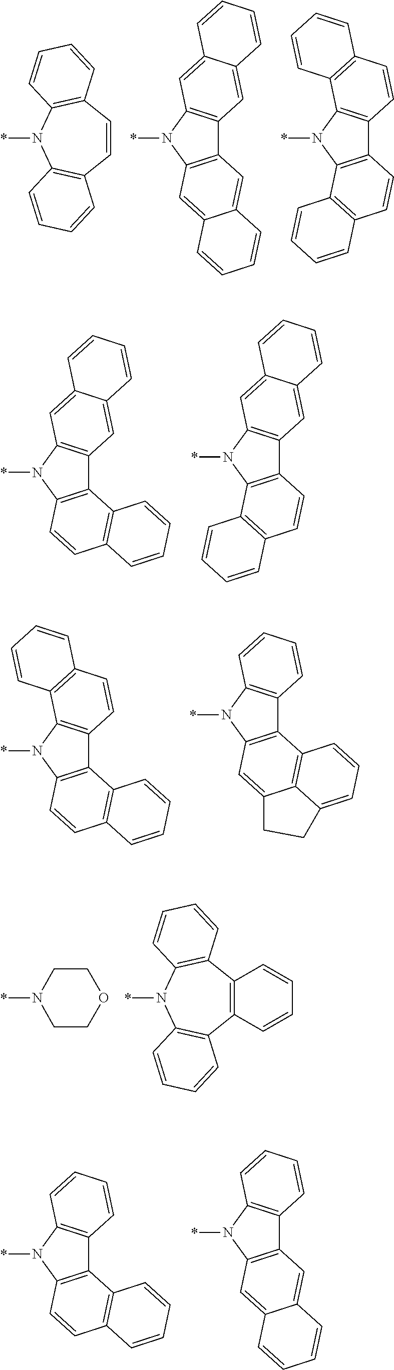

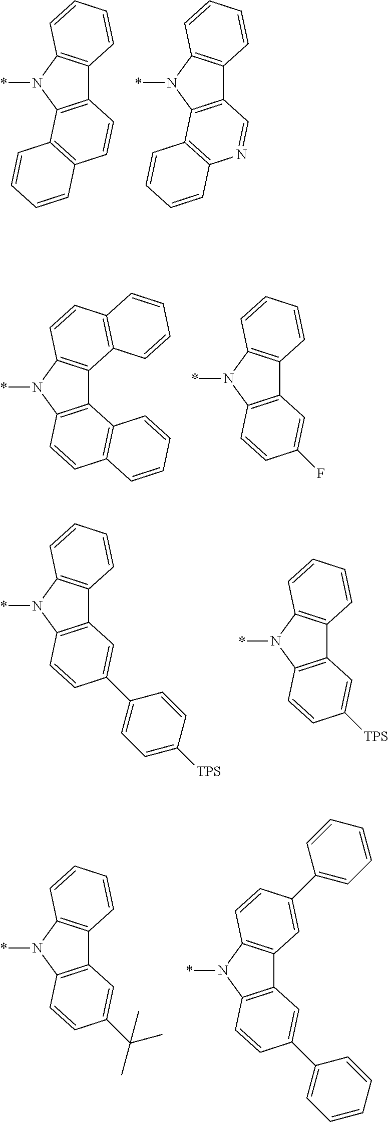

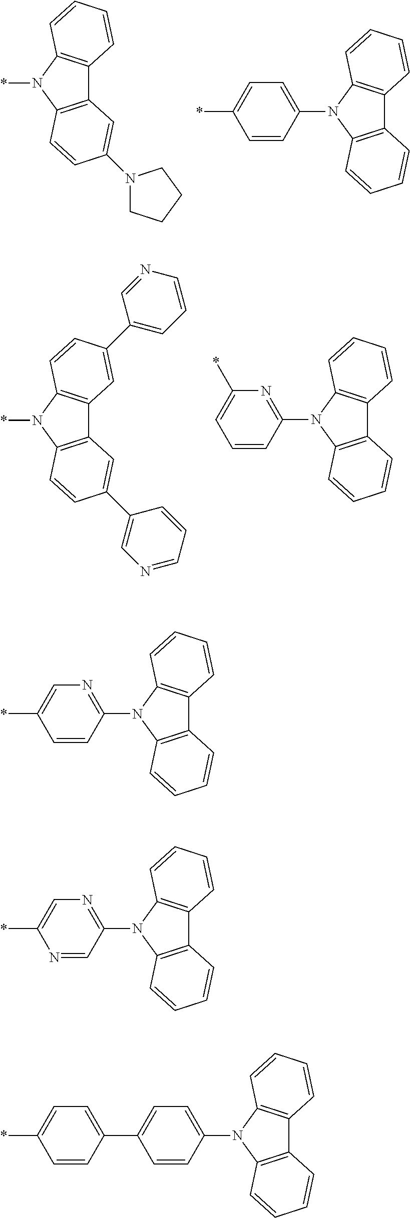

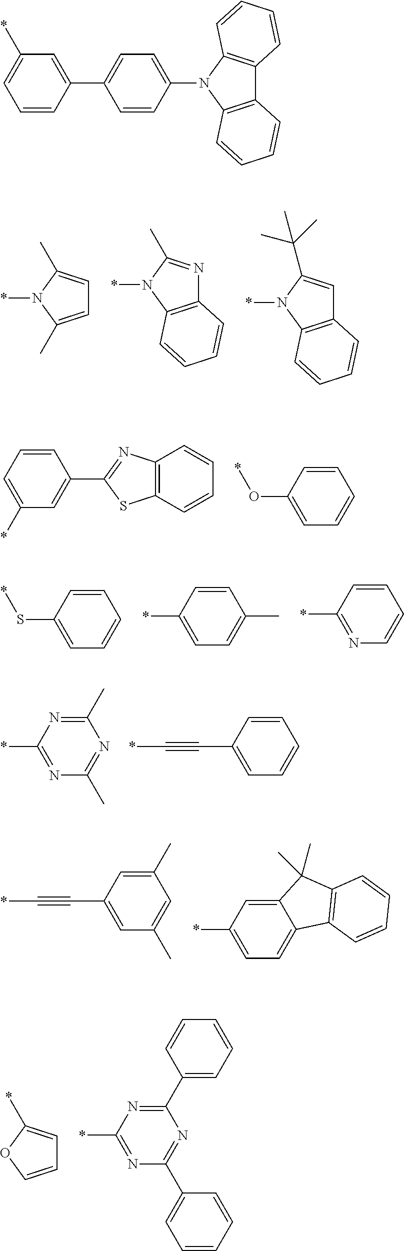

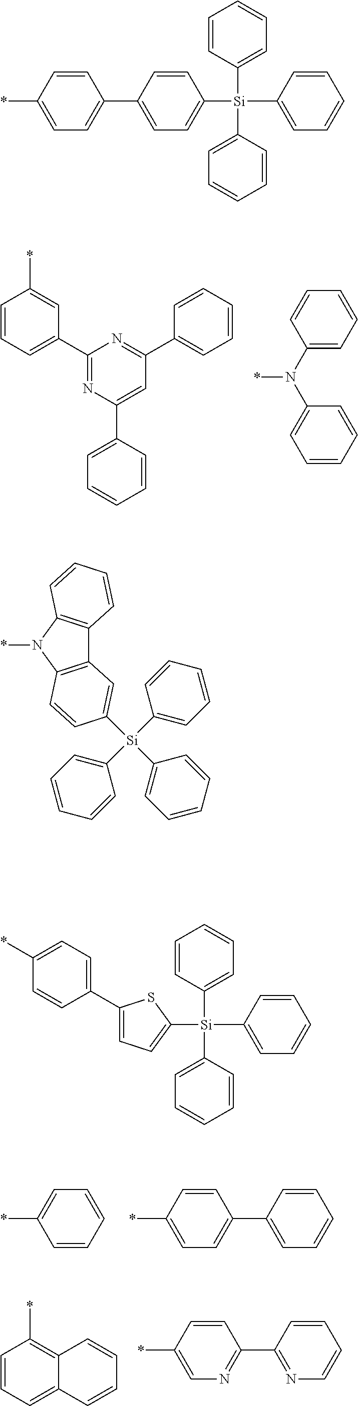

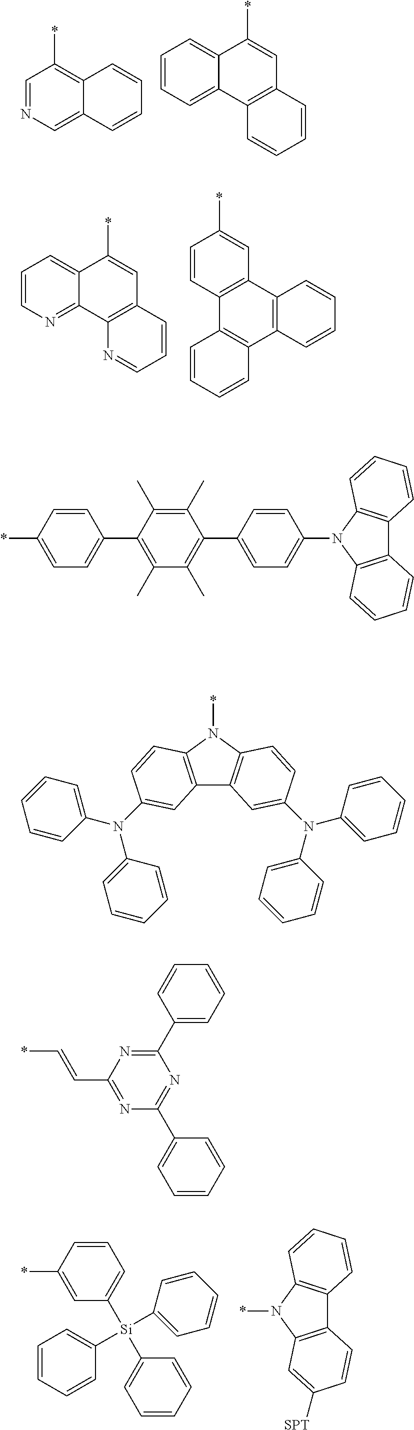

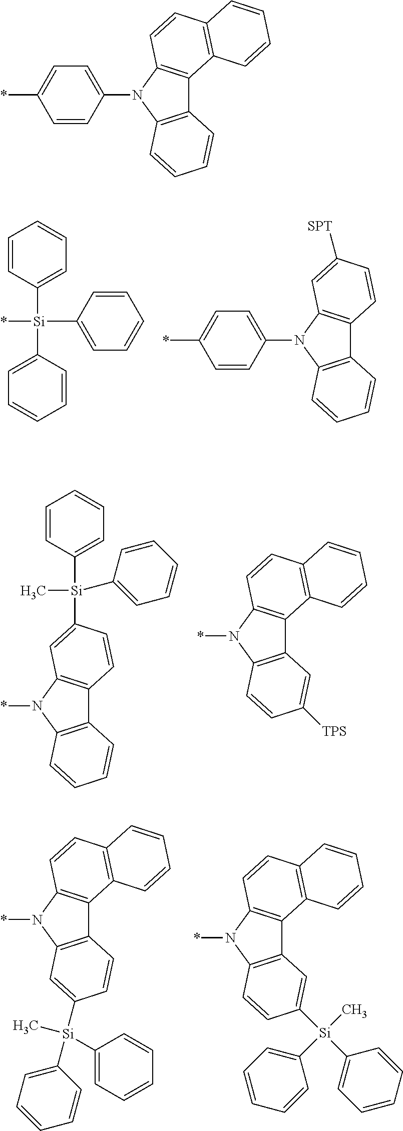

6. An organic electroluminescent device comprising the organic electroluminescent compound according to any of claims 1 to 5.

7. The organic electroluminescent device according to claim 6, which comprises a first electrode; a second electrode;
and one or more organic layer(s) interposed between the first electrode and the second electrode, wherein the organic layer comprises one or more organic electroluminescent compound(s) according to any of claims 1 to 5 and one or more phosphorescent dopant(s).

8. The organic electroluminescent device according to claim 7, wherein the organic layer further comprises one or more amine compound(s) selected from a group consisting of arylamine compounds and styrylarylamine compounds, or one or more metal(s) selected from a group consisting of organic metals of Group 1, Group 2; 4th period and 5th period transition metals, lanthanide metals and d-transition elements or complex compound(s).

9. The organic electroluminescent device according to claim 7, wherein the organic layer comprises an electroluminescent layer and a charge generating layer.

10. The organic electroluminescent device according to claim 7, which is a white light-emitting organic electroluminescent device wherein the organic layer comprises one or more organic electroluminescent layer(s) emitting blue, red or green light at the same time.
